SEQUENCE LISTING (1 sequence)

SEQ ID NO: 1 - DNA, 5477 bp, Artificial
  plasmid
tcgagctctccaatctccactgaggtacttaatccttccggggaattcgggcgcttaaatcgagaaattaggccatcaccttttaataacaatacaatgaataattggaataggtcgacacctttggagcggagccggttaaaattggcagcattcaccgaaagaaaaggagaaccacatgcttgccctaggttggattacatggatcattattggtggtctagctggttggattgcctccaagattaaaggcactgatgctcagcaaggaattttgctgaacatagtcgtcggtattatcggtggtttgttaggcggctggctgcttggaatcttcggagtggatgttgccggtggcggcttgatcttcagcttcatcacatgtctgattggtgctgtcattttgctgacgatcgtgcagttcttcactcggaagaagtaatctgctttaaatccgtagggcctgttgatatttcgatatcaacaggccttttggtcattttggggtggaaaaagcgctagacttgcctgtggattaaaactatacgaaccggtttgtctatattggtgttagacagttcgtcgtatcttgaaacagaccaacccgaaaggacgtggccgaacgtggctgctagctaatccttgatggtggacttgctggatctcgattggtccacaacatcagtcctcttgagacggctcgcgatttggctcggcagttgttgtcggctccacctgcggactactcaatttagtttcttcattttccgaaggggtatcttcgttgggggaggcgtcgataagccccttctttttagctttaacctcagcgcgacgctgctttaagcgctgcatggcggcgcggttcatttcacgttgcgtttcgcgcctcttgttcgcgatttctttgcgggcctgttttgcttcgttgatttcggcagtacgggttttggtgagttccacgtttgttgcgtgaagcgttgaggcgttccatggggtgagaatcatcagggcgcggtttttgcgtcgtgtccacaggaagatgcgcttttctttttgttttgcgcggtagatgtcgcgctgctctaggtggtgcactttgaaatcgtcggtaagtgggtatttgcgttccaaaatgaccatcatgatgattgtttggaggagcgtccacaggttgttgctgacgcgtcatatgactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

(19) United States
(12) Patent Application Publication
(10) Pub. No.: US 2010/0009416 A1
(43) Pub. Date: Jan. 14, 2010

(54) Process for the Preparation of L-Methionine

(76) Inventors: Oskar Zelder, Speyer (DE); Andrea Herold, Ketsch (DE); Corinna Klopprogge, Mannheim (DE); Hartwig Schröder, Nussloch (DE); Elmar Heinzle, Saarbrucken (DE); Christoph Wittmann, Braunschweig (DE); Jens Kroemer, Queensland (AU); Janice Pero, Lexington, MA (US); Rogers Yocum, Lexington, MA (US); Thomas Patterson, North Attleboro, MA (US); Mark Williams, Revere, MA (US); Theron Hermann, Kinnelon, NJ (US)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 12/301,279

(22) PCT Filed: May 24, 2007
(86) PCT No.: PCT/EP07/55056

§ 371 Date: Jun. 11, 2009

(30) Foreign Application Priority Data
May 24, 2006 (EP) 06114533.0

(51) Int. Cl. C12P 13/12; C12N 1/21

(52) U.S. Cl. 435/113; 435/252.3; 435/252.31; 435/252.32; 435/252.33; 435/252.34

(57) ABSTRACT

The present invention relates to microorganisms and processes for the efficient preparation of L-methionine. In particular, the present invention relates to processes in which the amount of serine available for the metabolism of the microorganism is increased.

### FIELD OF THE INVENTION

The present invention relates to microorganisms and processes for the efficient preparation of L-methionine. In particular, the present invention relates to processes in which the amount of serine available for the metabolism of the microorganism is increased.

### TECHNOLOGICAL BACKGROUND

Currently, the worldwide annual production of methionine is about 500,000 tons. Methionine is the first limiting amino acid in livestock of poultry feed and, due to this, mainly applied as feed supplement. In contrast to other industrial amino acids, methionine is almost exclusively applied as a racemate of D- and L-methionine which is produced by chemical synthesis. Since animals can metabolise both stereo-isomers of methionine, direct feed of the chemically produced racemic mixture is possible (D'Mello and Lewis, Effect of Nutrition Deficiencies in Animals: Amino Acids, Rechgigl (Ed.), CRC Handbook Series in Nutrition and Food, 441-490, 1978).

However, there is still a great interest in replacing the existing chemical production by a biotechnological process producing exclusively L-methionine. This is due to the fact that at lower levels of supplementation L-methionine is a better source of sulfur amino acids than D-methionine (Katz and Baker (1975) Poult. Sci. 545: 1667-74). Moreover, the chemical process uses rather hazardous chemicals and produces substantial waste streams. All these disadvantages of chemical production could be avoided by an efficient biotechnological process.

For other amino acids such as glutamate, it has been known to produce them using fermentation methods. For these purposes, certain microorganisms such as Escherichia coli (E. coli) and Corynebacterium glutamicum (C. glutamicum) have proven to be particularly suitable. The production of amino acids by fermentation also has the particular advantage that only L-amino acids are produced. Further, environmentally problematic chemicals such as solvents, etc. which are used in chemical synthesis are avoided. However, fermentative production of methionine by microorganisms will only be an alternative to chemical synthesis if it allows for the production of methionine on a commercial scale at a price comparable to that of chemical production.

Hence, the production of L-methionine by large-scale culture of bacteria developed to produce and secrete large quantities of this molecule is a desirable goal. Improvements of the process can relate to fermentation measures, such as stirring and supply of oxygen, or the composition of the nutrient media, such as the sugar concentration during fermentation, or the working up of the product by, for instance, ion exchange chromatography, or the intrinsic output properties of the microorganism itself.

Methods of mutagenesis and mutant selection are also used to improve the output properties of these microorganisms. High production strains which are resistant to antimetabolites or which are auxotrophic for metabolites of regulatory importance are obtained in this manner.

Recombinant DNA technology has also been employed for some years for improving microorganism strains which produce L-amino acids by amplifying individual amino acid biosynthesis genes and investigating their effect on the amino acid production.

Rückert et al. ((2003) Journal of Biotechnology 104: 213-228) provide an analysis of the L-methionine biosynthetic pathway in Corynebacterium glutamicum. Known functions of MetZ (also known as MetY) and MetB could be confirmed and MetC (also known as AecD) was proven to be a cystathionine-β-lyase. Further, MetE and MetH, which catalyse the conversion of L-homocysteine to L-methionine, were identified in this study.

WO 02/097096 uses nucleotide sequences from coryneform bacteria which code for the McbR repressor gene (also known as MetD) and processes for the preparation of amino acids using bacteria in which this McbR repressor gene is attenuated. According to WO 02/097096, the attenuation of the transcriptional regulator McbR improves the production of L-methionine in coryneform bacteria. It is further described in WO 02/097096 that, in addition to the attenuation of the McbR repressor gene, enhancing or overexpressing the MetB gene which codes for cystathionine-γ-synthase is preferred for the preparation of L-methionine.

Selection of strains improved for the production of a particular molecule is a time-consuming and difficult process. Therefore, there is still a great need for microorganisms which efficiently produce L-methionine and/or have significantly increased contents of L-methionine which can be utilized for obtaining the methionine compounds.

### SUMMARY OF THE INVENTION

It is an object of the present invention to provide methods for the efficient production of L-methionine in microorganisms.

It is a further object of the present invention to provide microorganisms which efficiently produce L-methionine.

These and further objects of the invention, as will become apparent from the description, are attained by the subject-matter of the independent claims.

Further embodiments of the invention are defined by the dependent claims.

According to one aspect of the invention a process for the preparation of L-methionine in a microorganism is provided, wherein the amount of serine available for the metabolism of the microorganism is increased.

This increase in the amount of serine available for the microorganism can be achieved by cultivating the microorganism in a medium which is enriched in serine.

The amount of serine available for the metabolism of the microorganism may also be increased by genetically modifying the microorganism.

Therefore, in one embodiment of the present invention, a process for the preparation of L-methionine in a microorganism is provided, wherein the microorganism is cultivated in a medium enriched in serine.

In another embodiment of the present invention, a process is provided wherein the microorganism is genetically modified with respect to proteins involved in serine metabolism or transport. This modification of the microorganism with respect to proteins involved in serine metabolism or transport can involve the increase of the content and/or the biological activity of one or more enzymes involved in serine synthesis, the decrease of the content and/or the biological activity of one or more enzymes involved in serine degradation to pyruvate, the increase of the content and/or the biological activity of one or more enzymes involved in the conversion of serine to methyl-tetrahydrofolate and/or the decrease of the content and/or the biological activity of one or more proteins involved in serine export from the cell.

According to a further embodiment of the process according to the present invention, the enzyme involved in serine synthesis is selected from the group consisting of D-3-phosphoglycerate dehydrogenase (SerA), phosphoserine phosphatase (SerB) and phosphoserine aminotransferase (SerC).

In a further preferred embodiment of the invention, the enzyme involved in serine synthesis is modified to reduce or prevent the feedback inhibition by L-serine.

According to a further embodiment of the process according to the present invention, the content and/or the biological activity of one or more enzymes involved in serine degradation to pyruvate is reduced compared to the wild-type microorganism. Preferably, the gene which codes for the enzyme involved in serine degradation is disrupted and most preferably the gene is eliminated. The enzyme involved in serine degradation is preferably sdaA.

In a further embodiment of the process of the present invention, the content and/or the biological activity of one or more proteins involved in serine export is reduced compared to the wild-type organism, preferably the gene which codes for the protein involved in serine export is disrupted, and most preferably the gene is eliminated. The protein involved in serine export is preferably ThrE.

In still a further embodiment of the process of the present invention, the content and/or the biological activity of one or more enzymes involved in the conversion of serine to methyl-tetrahydrofolate is increased compared to the wild-type microorganism. The enzyme involved in the conversion of serine to methyl-tetrahydrofolate is preferably selected from the group consisting of serine hydroxymethyltransferase and methylene tetrahydrofolate reductase.

In a preferred embodiment of the present invention, the content and/or the biological activity of one or more enzymes involved in methionine synthesis is increased compared to the wild-type organism in addition to increasing the amount of serine available for the metabolism of the microorganism by culturing the microorganism in a medium enriched in serine and/or genetically modifying a microorganism with respect to proteins involved in serine metabolism or transport.

Preferably, the enzyme involved in methionine synthesis is selected from the group consisting of homoserine-O-acetyltransferase (MetA), O-acetylhomoserine sulfhydrolase (MetZ), cob(I)alamin dependent methionine synthase I (MetH) and cob(I)alamin independent methionine synthase II (MetE), aspartokinase (lysC) and homoserine dehydrogenase (hom).

In another embodiment of the process of the present invention, the content and/or the biological activity of one or more transcriptional regulator proteins is reduced compared to the wild-type organism in addition to increasing the amount of serine available for the metabolism of the microorganism by culturing the microorganism in a medium enriched in serine and/or genetically modifying a microorganism with respect to proteins involved in serine metabolism or transport. Preferably, the transcriptional regulator protein is MbcR which, if present, represses the transcription of nucleic acid sequences encoding enzymes for methionine synthesis.

According to a further embodiment of the process of the present invention, the microorganism is selected from the group consisting of coryneform bacteria, mycobacteria, streptomycetaceae, salmonella, Escherichia coli, Shigella, Bacillus, Serratia and Pseudomonas.

According to a further embodiment of the process of the present invention, the desired L-methionine is concentrated in the medium or in the cells of the microorganism.

In a further aspect of the present invention, a process for the preparation of a L-methionine containing animal feedstuff additive from fermentation broths is provided which comprises the following steps:
preparing L-methionine in microorganisms in a process wherein the amount of serine available for the metabolism of the microorganism is increased;
removing water from the L-methionine containing fermentation broth;
removing an amount of 0 to 100 wt.-%, such as 10-90 wt.-% or 20-80 wt.-%, or 30-70 wt.-%, or 40-60 wt.-%, or about 50 wt.-% of the biomass formed during fermentation; and
drying the fermentation broth to obtain the animal feedstuffs additive in powder or granule form.

In a further embodiment of the present invention, an L-methionine over-producing microorganism is provided, wherein
the content and/or the biological activity of one or more enzymes involved in serine synthesis is increased compared to the wild-type microorganism; and/or
the content and/or the biological activity of one or more enzymes involved in serine degradation to pyruvate is reduced compared to the wild-type microorganism; and/or
the content and/or the biological activity of one or more proteins involved in serine export is reduced compared to the wild-type microorganism; and/or
the content and/or the biological activity of one or more enzymes involved in the conversion of serine to methyl-tetrahydrofolate is increased compared to the wild-type microorganism;
   and wherein
the content and/or the biological activity of one or more enzymes involved in methionine synthesis is increased compared to the wild-type microorganism; and/or
the content and/or the biological activity of one or more transcriptional regulator proteins is reduced compared to the wild-type microorganism.

Further, another aspect of the present invention relates to the use of a microorganism, in which the amount of serine is increased by genetically modifying the microorganism and which is genetically modified with respect to methionine synthesis, for the production of L-methionine.

### DESCRIPTION OF THE DRAWINGS

FIG. 1a is a model of the pathway for L-serine biosynthesis in microorganisms such as C. glutamicum. Enzymes involved in serine synthesis SerA (D-3-phosphoglycerate dehydrogenase), SerB (phosphoserine phosphatase) and SerC (phosphoserine aminotransferase). An enzyme involved in serine degradation is sdaA (serine dehydratase). Serine is converted to the methyl-donor methylene-tetrahydrofolate by the activity of glyA-shmt (serine hydroxymethyltransferase), which catalyses the transfer of a methylene group to tetrahydrofolate. In this reaction glycine is being produced as a side product.

FIG. 1b is a model of the pathway for L-methionine biosynthesis in microorganisms such as C. glutamicum. Enzymes involved are MetA (homoserine transacetylase), MetB (cystathionine-γ-synthase), MetZ (O-acetylhomoserine sulfhydrolase), MetC (cystathionine-β-lyase), cob(I)alamin dependent methionine synthase I (MetH) and cob(I)alamin independent methionine synthase II (MetE).

### DETAILED DESCRIPTION OF EXEMPLARY EMBODIMENTS

Before describing in detail exemplary embodiments of the present invention, the following definitions are given.

The term “efficiency of methionine synthesis” describes the carbon yield of methionine. This efficiency is calculated as a percentage of the energy input which entered the system in the form of a carbon substrate. Throughout the invention this value is given in percent values ((mol methionine) (mol carbon substrate)⁻¹×100) unless indicated otherwise.

The term “efficiency of serine synthesis” describes the carbon yield of serine. This efficiency is calculated as a percentage of the energy input which entered the system in the form of a carbon substrate. Throughout the invention this value is given in percent values ((mol serine) (mol carbon substrate)⁻¹×100) unless indicated otherwise.

Preferred carbon sources according to the present invention are sugars, such as mono-, di-, or polysaccharides. For example, sugars selected from the group consisting of glucose, fructose, mannose, galactose, ribose, sorbose, lactose, maltose, sucrose, raffinose, starch or cellulose may serve as particularly preferred carbon sources.

The term “increased efficiency of methionine synthesis” relates to a comparison between a microorganism that has been cultured in a medium enriched in serine and/or that has been genetically modified and which has a higher efficiency of methionine synthesis compared to the wild-type organism cultured under standard conditions.

The term “yield of methionine” describes the yield of methionine which is calculated as the amount of methionine obtained per weight cell mass.

The term “yield of serine” describes the yield of serine which is calculated as the amount of serine obtained per weight cell mass.

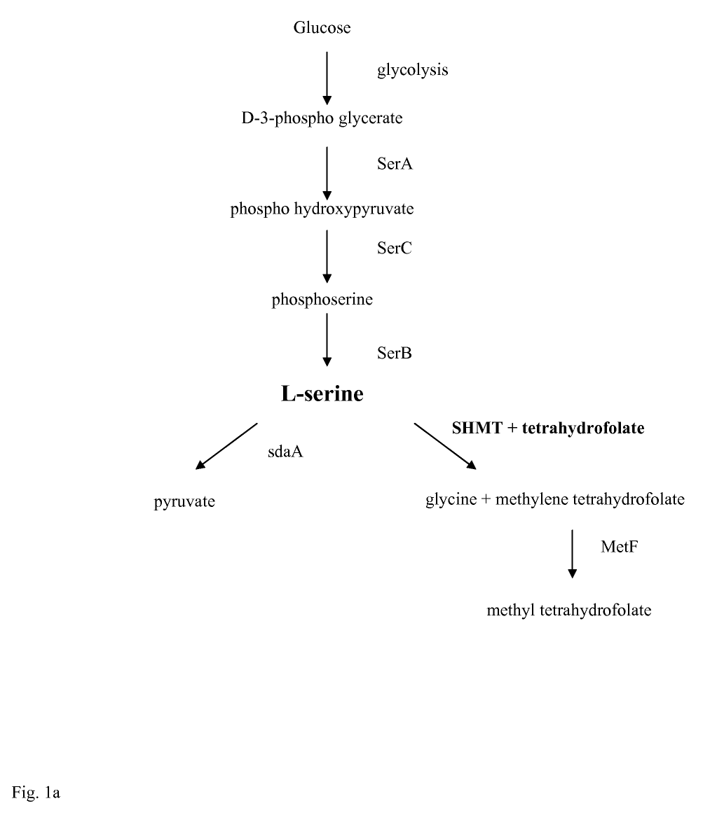

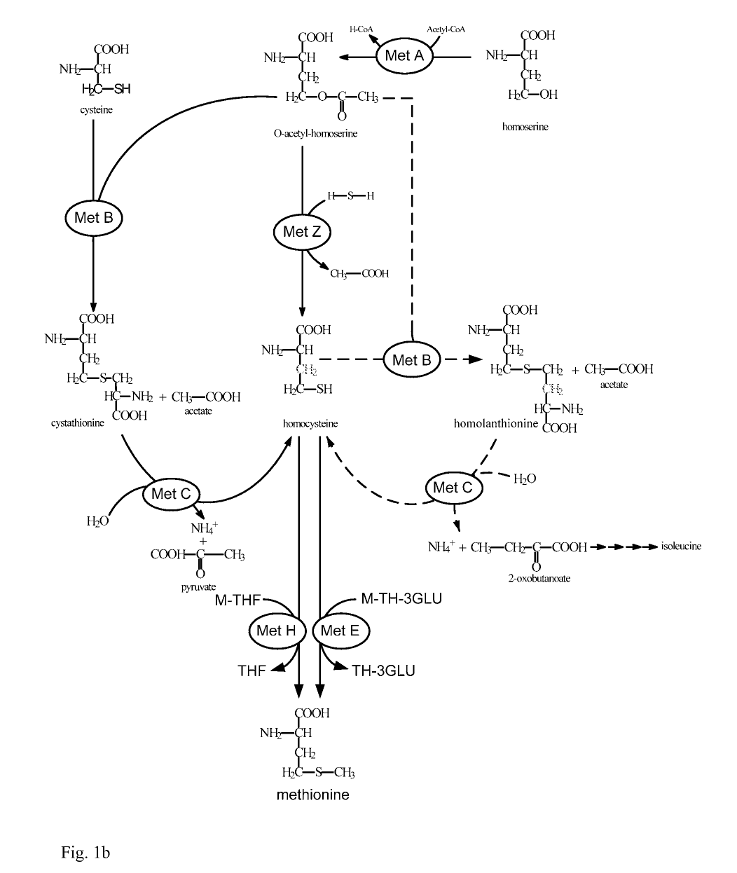

The term “methionine pathway” is art-recognized and describes a series of reactions which take place in a wild-type organism and lead to the biosynthesis of methionine. The pathway may vary from organism to organism. The details of an organism-specific pathway can be taken from textbooks and the scientific literature listed on the website http://www.genomejp/hegg/metabolism.html. In particular, a methionine pathway within the meaning of the present invention is shown in FIG. 1b.

The term “serine pathway” is art-recognized and describes a series of reactions which take place in a wild-type organism and lead to the biosynthesis of serine. The pathway may vary from organism to organism. The details of an organism-specific pathway can be taken from textbooks and the scientific literature listed on the website http://www.genome.jp. In particular, a serine pathway within the meaning of the present invention is shown in FIG. 1a.

The term “organism” or “microorganism” for the purposes of the present invention refers to any organism that is commonly used for the production of amino acids such as methionine. In particular, the term “organism” relates to prokaryotes, lower eukaryotes and plants. A preferred group of the above-mentioned organisms comprises actinobacteria, cyanobacteria, proteobacteria, Chloroflexus aurantiacus, Pirellula sp. 1, halobacteria and/or methanococci, preferably coryneform bacteria, mycobacteria, Streptomyces, Salmonella, Escherichia coli, Shigella and/or Pseudomonas. Particularly preferred microorganisms are selected from Corynebacterium glutamicum, Escherichia coli, microorganisms of the genus Bacillus, particularly Bacillus subtilis, and microorganisms of the genus Streptomyces.

The organisms of the present invention may, however, also comprise yeasts such as Schizosaccharomyces pombe, Saccharomyces cerevisiae and Pichia pastoris.

The term “L-methionine-overproducing microorganism” for the purposes of the present invention refers to a microorganism in which, compared to a wild-type microorganism cultured under standard conditions, the efficiency and/or yield and/or amount of methionine production is increased by at least 50%, at least 70%, 80% or 90%, at least 100%, at least 200%, at least 300%, 400% or 500%, at least 600%, at least 700% or 800%, at least 900% or at least 1000% or more.

Preferably, the microorganism is selected from the group consisting of coryneform bacteria, mycobacteria, streptomycetaceae, salmonella, Escherichia coli, Shigella, Bacillus, Serratia and Pseudomonas. More preferably, the microorganism is Escherichia coli or Corynebacterium glutamicum. Most preferably, the microorganism is Corynebacterium glutamicum.

The term “wild-type organism” or “wild-type microorganism” relates to an organism that has not been genetically modified.

The term “metabolite” refers to chemical compounds that are used in the metabolic pathways of organisms as precursors, intermediates and/or end products. Such metabolites may not only serve as chemical building units, but may also exert a regulatory activity on enzymes and their catalytic activity. It is known from the literature that such metabolites may inhibit or stimulate the activity of enzymes (Stryer, Biochemistry (2002) W.H. Freeman & Company, New York, N.Y.).

For the purposes of the present invention, the term “external metabolite” comprises substrates such as glucose, sulfate, thiosulfate, sulfite, sulfide, ammonia, oxygen, serine etc. In certain embodiments (external) metabolites comprise so called C1-metabolites. The latter metabolites can function as e.g. methyl donors and comprise compounds such as formate, formaldehyde, methanol, methanethiol, dimethyl-disulfid etc.

The term “products” comprises methionine, biomass, CO₂, etc.

Amino acids comprise the basic structural units of all proteins, and as such are essential for normal cellular functioning in organisms. The term “amino acid” is well known in the art. The proteinogenic amino acids, of which there are 20 species, serve as structural units for proteins, in which they are linked by peptide bonds, while the non-proteinogenic amino acids are not normally found in proteins (see Ullmann's Encyclopaedia of Industrial Chemistry, Vol. A2, pages 57-97, VCH, Weinheim (1985)). Amino acids may be in the D- or L-optical configuration, although L-amino acids are generally the only type found in naturally-occurring proteins. Biosynthetic and degradative pathways of each of the 20 proteinogenic amino acids have been well characterized in both prokaryotic and eukaryotic cells (see, for example, Stryer, L. Biochemistry, 5th edition (2002)).

The essential amino acids, i.e. histidine, isoleucine, leucine, lysine, methionine, phenylalanine, threonine, tryptophan and valine, which are generally a nutritional requirement due to the complexity of their biosynthesis, are readily converted by simple biosynthetic pathways to the 11 non-essential amino acids, i.e. alanine, arginine, asparagine, aspartate, cysteine, glutamate, glutamine, glycine, proline, serine and tyrosine. Higher animals do retain the ability to synthesize some of these amino acids, but the essential amino acids must be supplied from the diet in order for normal protein synthesis to occur. Apart from their function in protein biosynthesis, these amino acids are interesting chemicals in their own right, and many have been found to have various applications in the food, feed, chemical, cosmetic, agricultural and pharmaceutical industries. Lysine is an important amino acid in the nutrition not only of humans, but also of monogastric animals, such as poultry and swine. Glutamate is most commonly used as a flavour additive, and is widely used throughout the food industry as are aspartate, phenylalanine, glycine and cysteine. Glycine, L-methionine and tryptophan are all utilized in the pharmaceutical industry. Glutamine, valine, leucine, isoleucine, histidine, arginine, proline, serine and alanine are of use in both the pharmaceutical and cosmetic industries. Threonine, tryptophan and D/L-methionine are common feed additives (Leuchtenberger, W. (1996), Amino acids—technical production and use, p. 466-502 in Rehm et al. (editors) Biotechnology, Vol. 6, Chapter 14a, VCH: Weinheim). Additionally, these amino acids have been found to be useful as precursors for the synthesis of synthetic amino acids and proteins such as N-acetyl cysteine, S-carboxymethyl-L-cysteine, (S)-5-hydroxytryptophan and others described in Ullmann's Encyclopaedia of Industrial Chemistry, Vol. A2, p. 57-97, VCH: Weinheim, 1985.

The biosynthesis of natural amino acids in organisms capable of producing them, such as bacteria, has been well characterized (for review of bacterial amino acid biosynthesis and regulation thereof see Umbarger H. E. (1978) Ann. Rev. Biochem. 47: 533-606). Glutamate is synthesized by the reductive amination of α-ketoglutarate, an intermediate in the citric acid cycle. Glutamine, proline and arginine are each subsequently produced from glutamate. The biosynthesis of serine is a three-step process beginning with 3-phosphoglycerate (an intermediate in glycolysis), and resulting in this amino acid after oxidation, transamination, and hydrolysis steps. Both cysteine and glycine are produced from serine; the former by the condensation of homocysteine with serine, and the latter by transferal of the side-chain β-carbon atom to tetrahydrofolate, in a reaction catalyzed by serine hydroxymethyltransferase. Phenylalanine and tyrosine are synthesized from the glycolytic and pentose phosphate pathway precursors erythrose-4-phosphate and phosphoenolpyruvate in a nine-step biosynthetic pathway that differs only at the final two steps after the synthesis of prephenate. Tryptophan is also produced from these two initial molecules, but its synthesis is an eleven-step pathway. Tyrosine may also be synthesized from phenylalanine in a reaction catalysed by phenylalanine hydroxylase. Alanine, valine and leucine are all biosynthetic products of pyruvate, the final product of glycolysis. Aspartate is formed from oxaloacetate, an intermediate of the citric acid cycle. Asparagine, methionine, threonine and lysine are each produced by the conversion of aspartate. Isoleucine may be formed from threonine. A complex nine-step pathway results in the production of histidine from 5-phosphoribosyl-1-pyrophosphate, an activated sugar.

Amino acids in excess of the protein synthesis needs of the cell cannot be stored and are instead degraded to provide intermediates for the major metabolic pathways of the cell (for review see Stryer, L., Biochemistry, 5th edition (2002), Chapter 23 “Protein Turnover: Amino acid degradation and the urea cycle”). Although the cell is able to convert excess amino acids into useful metabolic intermediates, amino acid production is costly in terms of energy, precursor molecules, and the enzymes necessary to synthesise them. Thus, it is not surprising that amino acid biosynthesis is regulated by feedback inhibition, in which the presence of a particular amino acid serves to slow or entirely stop its own production (for an overview of feedback mechanisms in amino acid biosynthetic pathways see Stryer, L., Biochemistry, 5th edition (2002), Chapter 24: “Biosynthesis of amino acids”). Thus, the output of any particular amino acid is limited by the amount of that amino acid present in the cell.

The Gram-positive soil bacterium Corynebacterium glutamicum is widely used for the industrial production of different amino acids. In contrast to lysine and glutamate, the main industrial products, the biosynthesis of which has been studied for many years, knowledge about the regulation of the methionine biosynthetic pathway is limited. At least the key enzymes of the pathway are known (see FIG. 1b). Homoserine is produced from aspartate by three subsequent reactions catalyzed by aspartokinase (lysC), β-aspartate semialdehyde dehydrogenase and homoserine dehydrogenase (hom). C. glutamicum activates homoserine by acetylation with homoserine-O-acetyltransferase (MetA) (EC 2.3.1.31). It was further shown that both transsulfuration and direct sulfhydrylation are used to produce homocysteine (Hwang, B. J. et al (2002) J. Bacteriol. 184(5): 1277-86). Transsulfuration is catalyzed by cystathionine-γ-synthase (MetB) (EC 2.5.1.48) (Hwang, B. J. et al. (1999) Mol Cells 93: 300-8). In this reaction, cysteine and O-acetyl-homoserine are combined to cystathionine, which is hydrolyzed by the cystathionine-β-lyase (MetC, which is also known as AecD) (EC 4.4.1.8) (Kim, J. W. et al (2001) Mol Cells 112: 220-5; Ruckert et al. (2003) vide supra) to homocysteine, pyruvate and ammonia. In the direct sulfhydrylation O-acetylhomoserine sulfhydrolase (MetZ, which is also known as MetY) (EC 2.5.1.49) (Ruckert et al. (2003) vide supra) converts O-acetylhomoserine and sulfide into homocysteine and acetate. Finally, C. glutamicum has two different enzymes for the S-methylation of homocysteine yielding methionine (Lee, H. S. and Hwang, B. J. (2003) Appl. Microbiol. Biotechnol. 625-6: 459-67; Ruckert et al., 2003, vide supra), i.e. a cob(I)alamin dependent methionine synthase I (MetH) (EC 2.1.1.13) and a cob(I)alamin independent methionine synthase II (MetE) (EC 2.1.1.14). The former utilizes 5-methyltetrahydrofolate and the latter 5-methyltetrahydropteroyltri-L-glutamate as the methyl donor.

Recently, a putative transcriptional regulator protein of the TetR-family was found (Rey et al. (2003) Journal of Biotechnology 103: 51-65). This regulator was shown to repress the transcription of several genes encoding enzymes of the methionine and sulfur metabolism. The gene knockout of the regulator protein led to an increased expression of hom encoding homoserine dehydrogenase, metZ encoding O-acetylhomoserine sulfhydrolase, metK encoding S-adenosylmethionine (SAM) synthase (EC 2.5.1.6), cysK encoding cysteine synthase (EC 2.5.1.47), cysI encoding a putative NADPH-dependent sulfite reductase, and finally ssuD encoding a putative alkanesulfonate monooxygenase. Rey et al. (Molecular Microbiology (2005) 56: 871-887) also found that the metB gene is significantly induced in a mcbR minus strain.

Serine is synthesized from the glycolytic intermediate 3-phosphoglycerate which is first oxidized to phosphohydroxypyruvate by the action of 3-phosphoglycerate dehydrogenase (SerA; EC: 1.1.1.95). In a second step, transamination of phosphohydroxypyruvate catalyzed by phosphoserine aminotransferase (SerC; EC: 2.6.1.52) leads to the formation of phosphoserine, which is subsequently dephosphorylated by phosphoserine phosphatase (SerB; EC: 3.1.3.3) to yield L-serine. L-serine can be converted to pyruvate by the serine dehydratase sdaA (EC: 4.3.1.17) and to glycine and methylene tetrahydrofolate by serine hydroxymethyltransferase (SHMT; EC 2.1.2.1) (see FIG. 1a). Methylene-tetrahydrofolate can be converted to methyl-tetrahydrofolate by the activity of Methylene-tetrahydrofolate reductase metF (EC 1.5.1.20).

It has now surprisingly been found that increasing the amount of serine which is available for the metabolism of the microorganism leads to an increase in the production of L-methionine in the microorganism.

The present invention is based on the discovery that supplying a microorganism with an increased amount of serine leads to an increase in the synthesis of methionine and therefore the efficiency of synthesis and/or the yield of L-methionine is increased. The amount of serine which is available for the metabolism of the microorganism may be increased by cultivating the microorganism in a medium enriched in serine. In addition or alternatively, the microorganism may be genetically modified with respect to proteins involved in serine metabolism or transport. If a microorganism genetically modified with respect to proteins involved in serine metabolism or transport is cultivated in a medium enriched in serine, this may have an even greater effect on methionine yield.

The term “metabolism” is intended to comprise all biochemical reactions that occur within the cell and which lead to the synthesis and degradation of molecules.

The term “increase in the amount serine” refers to an increase in the amount of serine which can be used by the microorganism for the synthesis of biomolecules compared to a wild-type microorganism cultured under standard culture conditions by at least 10%, at least 20%, at least 30%, preferably at least 40%, preferably at least 50%, more preferably at least 60% and 70%, even more preferably at least 80% and 90%, particularly preferred at least 100%, 110% and 120%, and most preferably at least 160%, 200% and 250%. The amount of serine within a cell can be determined as described by Wittmann et al. ((2004) Anal. Biochem. 327(1): 135-139)

The term “standard conditions” refers to the cultivation of a microorganism in a standard medium which is not enriched in serine. The temperature, pH and incubation time can vary as described below.

The standard culture conditions for each microorganism used can be taken from the textbooks, such as Sambrook and Russell, Molecular Cloning—A laboratory manual, Cold Spring Harbour Laboratory Press, 3^{rd} edition (2001).

E.g., E. coli and C. glutamicum strains are routinely grown in MB or LB and BHI broth (Follettie, M. T. et al. (1993) J. Bacteriol. 175: 4096-4103, Difco Becton Dickinson). Usual standard minimal media for E. coli are M9 and modified MCGC (Yoshihama et al. (1985) J. Bacteriol. 162: 591-507; Liebl et al. (1989) Appl. Microbiol. Biotechnol. 32: 205-210.). Other suitable standard media for the cultivation of bacteria include NZCYM, SOB, TB, CG12½ and YT.

“Standard media” within the meaning of the present invention are intended to include all media which are suitable for the cultivation of the microorganisms of the present invention. Both enriched and minimal media are comprised with minimal media being preferred. Standard media within the context of the present invention do not comprise media to which one or more amino acids have been added or are added.

“Minimal media” are media that contain only the minimal necessities for the growth of wild-type cells, i.e. inorganic salts, a carbon source and water.

In contrast, “enriched media” are designed to fulfil all growth requirements of a specific microorganism, i.e. in addition to the contents of the minimal media they contain for example growth factors.

Antibiotics may be added to the standard media in the following amounts (micrograms per milliliter): ampicillin, 50; kanamycin, 25; nalidixic acid, 25 to allow for the selection of transformed strains.

Genetically modified Corynebacteria are typically cultured in synthetic or natural growth media. A number of different growth media for Corynebacteria are both well-known and readily available (Lieb et al. (1989) Appl. Microbiol. Biotechnol., 32: 205-210; von der Osten et al. (1998) Biotechnology Letters 11: 11-16; Liebl (1992) “The Genus Corynebacterium, in: The Procaryotes, Volume II, Balows, A. et al., eds. Springer-Verlag). Examples for C. glutamicum vectors can be found in the Handbook of Corynebacterium (Eggeling, L. Bott, M., eds., CRC press USA 2005).

Suitable media consist of one or more carbon sources, nitrogen sources, inorganic salts, vitamins and trace elements. Preferred carbon sources are sugars, such as mono-, di-, or polysaccharides. For example, glucose, fructose, mannose, galactose, ribose, sorbose, lactose, maltose, sucrose, raffinose, starch or cellulose may serve as very good carbon sources.

It is also possible to supply sugar to the media via complex compounds such as molasses or other by-products from sugar refinement. It can also be advantageous to supply mixtures of different carbon sources. Other possible carbon sources are alcohols and organic acids, such as methanol, ethanol, acetic acid or lactic acid. Nitrogen sources are usually organic or inorganic nitrogen compounds, or materials which contain these compounds. Exemplary nitrogen sources include ammonia gas or ammonia salts, such as NH₄Cl or (NH₄)₂SO₄, NH₄OH, nitrates, urea, amino acids or complex nitrogen sources like corn steep liquor, soy bean flour, soy bean protein, yeast extract, meat extract and others.

The overproduction of methionine is possible using different sulfur sources. Sulfates, thiosulfates, sulfites and also more reduced sulfur sources like H₂S and sulfides and derivatives can be used. Also organic sulfur sources like methyl mercaptan, thioglycolates, thiocyanates, thiourea, sulfur-containing amino acids like cysteine and other sulfur-containing compounds can be used to achieve efficient methionine production. Formate and/or methanethiol may also be possible as a supplement as are other C1 sources such as formaldehyde, methanol and dimethyl-disulfide.

Inorganic salt compounds which may be included in the media include the chloride, phosphate or sulfate salts of calcium, magnesium, sodium, cobalt, molybdenum, potassium, manganese, zinc, copper and iron. Chelating compounds can be added to the medium to keep the metal ions in solution. Particularly useful chelating compounds include dihydroxyphenols, like catechol or protocatechuate, or organic acids, such as citric acid. It is typical for the media to also contain other growth factors, such as vitamins or growth promoters, examples of which include biotin, riboflavin, thiamin, folic acid, nicotinic acid, pantothenate and pyridoxin. Growth factors and salts frequently originate from complex media components such as yeast extract, molasses, corn steep liquor and others. The exact composition of the media compounds depends strongly on the immediate experiment and is individually decided for each specific case. Information about media optimization is available in the textbook “Applied Microbiol. Physiology, A Practical Approach (eds. P. M. Rhodes, P. F. Stanbury, IRL Press (1997) pp. 53-73, ISBN 0 19 963577 3). It is also possible to select growth media from commercial suppliers, like standard 1 (Merck) or BHI (brain heart infusion, DIFCO) or others.

All medium components should be sterilized, either by heat (20 minutes at 1.5 bar and 121° C.) or by sterile filtration. The components can either be sterilized together or, if necessary, separately.

The preparation of standard media used for the cultivation of bacteria usually does not involve the addition of single amino acids. Instead, in enriched media for use under standard culture conditions a mixture of amino acids such as peptone or trypton is added. Therefore, an enrichment of the medium with serine in accordance with the present invention is achieved by additionally adding pure serine in a defined concentration to the standard medium as described above. Preferably, the concentration of serine added to the medium is from 0.1 mM to 100 mM, preferably from 1 to 50 mM, more preferably from 5 mM to 20 mM and most preferably the concentration of serine is 10 mM. It is also possible to feed a serine stock solution to a continuously feed fermentation in the way that the current concentration of serine is being kept between 0.1 mM serine and 10 mM, preferably between 0.1 mM to 5 mM and most preferably between 0.1 mM to 1 mM.

The increase in the amount of serine available for the metabolism of the microorganism can also be achieved by genetically modifying the microorganism with respect to proteins involved in serine metabolism or transport.

The term “genetically modified” within the meaning of the present invention is intended to mean that the microorganism has been modified by means of gene technology to express an altered amount of one or more proteins, which can be naturally present in the wild-type organism or which are not naturally present in the wild-type microorganism, or proteins with an altered activity in comparison to the proteins of the wild-type microorganism.

The term “serine metabolism” is intended to comprise all reactions leading to serine synthesis and serine degradation.

The term “serine transport” is intended to mean the import and export of serine into the cell or out of the cell, respectively, by means of specific transport proteins.

With respect to increasing or decreasing the content or amount and/or biological activity of a protein, all methods that are known in the art for increasing or decreasing the amount and/or activity of a protein in a host such as the above-mentioned organisms may be used.

The amount of the protein may be increased by expression of an exogenous version of the respective protein. Further, expression of the endogenous protein can be increased by influencing the activity of the promoter and/or enhancer elements and/or other regulatory activities such as phosphorylation, isoprenylation etc. that regulate the activities of the respective proteins either on a transcriptional, translational or post-translational level.

Besides simply increasing the amount of one or more proteins, the activity of the proteins may be increased by using enzymes which carry specific mutations that allow for an increased activity of the enzyme. Such mutations may, e.g. inactivate the regions of an enzyme that are responsible for feedback inhibition. By mutating these by e.g. introducing non-conservative mutations, the enzyme does not provide for feedback regulation anymore and thus the activity of the enzyme is not down-regulated if more product molecules are produced. The mutations may be either introduced into the endogenous copy of the enzyme, or may be provided by over-expressing a corresponding mutant form of the exogenous enzyme. Such mutations may comprise point mutations, deletions or insertions. Point mutations may be conservative (replacement of an amino acid with a biochemically similar one) or non-conservative (replacement of an amino acid with another which is not biochemically similar). Furthermore, deletions may comprise only two or three amino acids up to complete domains of the respective protein.

Examples of suitable mutations within the D-3-phosphoglycerate dehydrogenase enzyme which abolish the feedback regulation of the enzyme by L-serine can be derived from the literature, e.g. Bell et al. (2002) Eur. J. Biochem. 269: 4176-4184; Al-Rabiee et al. (1996) J. Biol. Chem. 271(38): 23235-23238; Peters-Wendisch et al. (2005) Appl. Environ. Microbiol. 71(11): 7139-7144. These articles are herein incorporated by reference.

Thus, the increase of the amount and/or the activity of a protein may be achieved via different routes, e.g. by switching off inhibitory regulatory mechanisms at the transcription, translation, or protein level or by increase of gene expression of a nucleic acid coding for these proteins in comparison with the wild type, e.g. by inducing the endogenous gene or by introducing nucleic acid molecules coding for the protein.

In one embodiment, the increase of the enzymatic activity and amount, respectively, in comparison with the wild type is achieved by an increase of the gene expression of a nucleic acid encoding enzymes such as SerA [EC: 1.1.1.95], SerB [EC: 3.1.3.3] and SerC [EC: 2.6.1.52], SHMT [EC1.2.1.2], metF [EC 1.5.1.20], or by a decrease of the gene expression of a nucleic acid encoding proteins such as sdaA [EC: 4.3.1.17] and ThrE. Nucleic acid sequences coding for these proteins may be obtained from the respective database, e.g. at NCBI (http://www.ncbi.nlm.nih.gov/), EMBL (http://www.embl.org), Expasy (http://www.expasy.org/), KEGG (http://www.genome.ad.jp/kegg/kegg.html) etc. Examples are given in Table 1.

TABLE 1

| Name | Enzyme | Gene bank accession number | Organism |
|---|---|---|---|
| SerA | D-3-phosphoglycerate | NCgl1235, CE1379, DIP1104, | C. glutamicum and |
| | dehydrogenase | jk1291, nfa42210, MAP3033c, | others |
| | | Mb3020c, MT3074, Rv2996c, | |
| | | ML1692, Tfu_0614, SAV2730, | |
| | | SCO5515, Francci3_3637, | |
| | | Lxx13140, CC3215, | |
| | | Jann_0261, CHY_2698, | |
| | | MMP1588, VNG2424G, | |
| | | RSP_1352, CYB_1383, | |
| | | AGR_L_2264, Atu3706, | |
| | | ZMO1685, tlr0325, NP0272A, | |
| | | Mbur_2385, Moth_0020, | |
| | | Adeh_1262, SMc00641, | |
| | | RHE_CH03454, rrnAC2696, | |
| | | MJ1018, TTE2613, amb3193, | |
| | | AF0813, MK0297, DET0599, | |
| | | CYA_1354, Synpcc7942_1501, | |
| | | syc2486_c, Saro_2680, | |
| | | ELI_01970, MM1753, | |
| | | cbdb_A580, BR1685, MTH970, | |
| | | Mbar_A1431, SPO3355, | |
| | | BruAb1_1670, BAB1_1697, | |
| | | BMEI0349, SYNW0533, | |
| | | Syncc9605_2150, Ava_3759, | |
| | | MA0592, alr1890, Mhun_3063, | |
| | | Syncc9902_0527, RPB_1315, | |
| | | glr2139, RPD_3905, | |
| | | Nwi_2968, RPA4308, | |
| | | SYN_00123, ABC1843, | |
| | | Nham_1119, STH9, bll7401, | |
| | | sll1908, CTC00694, BH1602, | |
| | | GK2247, RPC_4106, SH1200, | |
| | | Pcar_3115, Gmet_2378, | |
| | | SSP1039, BLi02446, BL00647, | |
| | | OB2626, BG10509, | |
| | | Acid345_0115, Dgeo_0710, | |
| | | Pro1436, SAR1801, SAB1582, | |
| | | SAV1724, SA1545, SERP1288, | |
| | | SE1401, SAS1650, MW1666, | |
| | | SAOUHSC_01833, | |
| | | SAUSA300_1670, | |
| | | SACOL1773, mll3875, | |
| | | GSU1198, HH0135, WS1313, | |
| | | Tmden_0875, PMT1431, | |
| | | DR1291, PMT9312_1452, | |
| | | TTC0586, Msp_1145, | |
| | | At1g17745, TTHA0952, | |
| | | PMM1354, At4g34200, | |
| | | RB6248, PMN2A_0926, | |
| | | CJE0970, Cj0891c, Pcar_0417, | |
| | | CMC149C, At3g19480, | |
| | | aq_1905, jhp0984, HP0397, | |
| | | PH1387, PAB0514, TK1966, | |
| | | C31C9.2, PF1394, Cag_1377, | |
| | | TM1401, Afu2g04490, | |
| | | CG6287-PA, rrnAC1762, | |
| | | AGR_pAT_578, PF0319, | |
| | | Atu5399, PAB2374, OB2286, | |
| | | Adeh_1858, BLi03698, | |
| | | BL03435, TK0683, PH0597, | |
| | | Reut_B3530, GK1954, | |
| | | ABC0220, MK0320, DSY0969, | |
| | | BP0155, Bxe_B1896, BB4474, | |
| | | BPP4001, STH3215, OB2844, | |
| | | CAC0015, RPC_3076, | |
| | | rrnAC2056, RPC_1162, | |
| | | AGR_pAT_470, Atu5328, | |
| | | PP3376, PAE3320, Bd1461, | |
| | | Pfl_2987, Rmet_4234, | |
| | | CNA07520, GK2965, MS1743, | |
| | | VV11546, LA1911, mll1021, | |
| | | MS0068, lp_0785, lin0070, | |
| | | VV2851, ebA6869, RPA2975, | |
| | | Tcr_0627, LIC11992, | |
| | | TTE1946, MA1334, | |
| | | LMOf2365_0095, Sde_3388, | |
| | | lmo0078, LmjF03.0030, | |
| | | SH0752, Rmet_4537, | |
| | | orf19.5263, VP2593, BCE1535, | |
| | | RPD_2906, CPE0054, OB2357, | |
| | | bll7965, BAS1325, BA_1955, | |
| | | GBAA1434, BA1434, | |
| | | Reut_B4747, PFL_2717, | |
| | | PA2263, YPTB3189, YP3611, | |
| | | y3301, YPO0914, GOX0218, | |
| | | ACL032C, RSP_3407, | |
| | | VC2481, BT9727_1298, | |
| | | BCZK1299, BMEII0813, | |
| | | BTH_I2298, Reut_B4615, | |
| | | ECA3905, YPTB3910, | |
| | | YP3988, YPO4078, RPB_2550, | |
| | | BruAb2_0769, BRA0453, | |
| | | BAB2_0783, Pfl_2904, | |
| | | plu3605, PAE1038, DSY1673, | |
| | | Sden_3097, NTHI0596, | |
| | | SERP1888, blr4558, | |
| | | Rfer_1867, YER081W, | |
| | | BC1415, Pcar_0629, VF2106, | |
| | | y4096, SPCC4G3.01, SE1879, | |
| | | SAR2389, BB4731, Psyc_0369, | |
| | | TK0551, SCO3478, Csal_1770, | |
| | | XCV1890, Bcep18194_A5027, | |
| | | PM1671, SAOUHSC_02577, | |
| | | SAUSA300_2254, | |
| | | SACOL2296, SAS2196, | |
| | | MW2224, BLi03415, BL02138, | |
| | | Mfla_0724, PSPTO5294, | |
| | | XOO3260, XC_1568, | |
| | | XCC2550, SAB2178, | |
| | | SAV2305, SA2098, | |
| | | PSPPH_4885, XCV2876, | |
| | | SH2023, Adeh_2960, | |
| | | BURPS1710b_2286, | |
| | | BPSL1577, Pcryo_0410, | |
| | | NE1688, YPTB1320, YP1303, | |
| | | t2980, STY3218, Mbar_A2220, | |
| | | Psyr_4852, HI0465, y2896, | |
| | | YPO1288, STM3062, | |
| | | SPA2933, YIL074C, | |
| | | SERP0516, Bxe_A1982, | |
| | | XAC2724, SC3003, BB1050, | |
| | | Afu5g05500, SSP0606, | |
| | | SG2009, SE0622, XCC1825, | |
| | | SBO_2700, PF0370, | |
| | | SBO_3080, SSO_3065, S3098, | |
| | | SF2898, UTI89_C3299, c3494, | |
| | | ECs3784, Z4251, JW2880, | |
| | | b2913, SRU_0653, SAB0796, | |
| | | SAR0892, Bd2892, | |
| | | ACIAD3302, Saci_1368, | |
| | | SSP1845, Bcep18194_A4216, | |
| | | Psyr_1043, Csal_0273, | |
| | | PPA2251, DVU0339, | |
| | | PFL_5911, SDY_3169, | |
| | | DDB0230052, SAS0800, | |
| | | MW0812, IL2104, PA4626, | |
| | | XC_2364, SAUSA300_0834, | |
| | | SACOL0932, SAV0930, | |
| | | SA0791, Bpro_1736, | |
| | | SMc01622, amb0136, | |
| | | PSPPH_1099, XOO2143, | |
| | | XAC1844, PAB1008, RB6394, | |
| | | LBA0942, MCA1407, | |
| | | PSPTO1215, PH0520, | |
| | | TM0327, SAOUHSC_00866, | |
| | | BG12409, Reut_A2281, | |
| | | ELI_06720, SMc01943, | |
| | | SDY_4350, TTC0431, all8087, | |
| | | GSU1672, Nmul_A0428, | |
| | | BTH_I2885, | |
| | | BURPS1710b_1481, | |
| | | BPSL1250, Ta0779, DSY4020, | |
| | | BLi03716, BL03603, amb0195, | |
| | | RSP_3447, UTI89_C4093, | |
| | | ECs4438, Z4978, PSHAa0666, | |
| | | PFL_1001, SBO_3555, | |
| | | Rru_A2456, Dde_1681, | |
| | | BTH_I1700, Pfl_5387, | |
| | | XF2206, S4182, SF3587, | |
| | | c4372, Reut_C5898, | |
| | | CPS_2082, SSO_3835, | |
| | | VNG0104G, TTHA0786, | |
| | | Pfl_2771, APE1831, SO0862, | |
| | | PD1255, ST1218, Moth_1954, | |
| | | BB1529, Csal_0096, SAV7481, | |
| | | Bxe_A1055, PP5155, | |
| | | UTI89_C3212, CG1236-PA, | |
| | | SSO0905, SAK_1826, gbs1847, | |
| | | SAG1806, blr3173, PA0316, | |
| | | ECA0078, DDB0231445, | |
| | | SMa2137, JW5656, b3553, | |
| | | GOX0065, | |
| | | BURPS1710b_2926, | |
| | | BPSL2459, BMA0513, | |
| | | Rmet_2446, | |
| | | SAOUHSC_00142, | |
| | | SAUSA300_0179, | |
| | | SACOL0162, SAS0152, | |
| | | SAR0178, MW0151, | |
| | | SAV0177, SA0171, BPP2132, | |
| | | RSc1034, PP1261, c3405, | |
| | | Dde_3689, CAC0089, | |
| | | SMc02849, mlr7269, PTO0372, | |
| | | BR2177, RSc3131, Mb0749c, | |
| | | MT0753, Rv0728c, DSY3442, | |
| | | SAB0117, Gmet_2695, | |
| | | Noc_2032, SC3578, | |
| | | BruAb1_2150, BAB1_2178, | |
| | | BMEI1952, BTH_I1402 | |
| SerB | phosphoserine phospatase | NCgl2436, cg2779, CE2417, | C. glutamicum and |
| | | DIP1863, jk0483, nfa42930, | others |
| | | MAP3090c, ML1727, | |
| | | Mb3068c, MT3127, Rv3042c, | |
| | | SCO1808, SAV6470, | |
| | | Tfu_0136, CT0173, Psyr_0557, | |
| | | PSPTO4957, PP4909, | |
| | | Sde_1075, HCH_05403, | |
| | | Plut_1948, PSPPH_0550, | |
| | | PA4960, ACIAD3567, | |
| | | Pfl_0506, Pcar_2283, BF2389, | |
| | | BF2300, RB8037, Cag_0409, | |
| | | PFL_0551, PG0653, BT0832, | |
| | | CMI086C, Csal_2542, | |
| | | Acid345_2803, AF2138, | |
| | | Lxx11750, Rmet_1368, | |
| | | Psyc_1857, AO090020000345, | |
| | | Afu3g06550, SPBC3H7.07c, | |
| | | Pcryo_2146, SMU.1269, | |
| | | stu1519, str1519, Reut_A1357, | |
| | | PBPRA0635, Mfla_1890, | |
| | | Rru_A0465, ACL130C, | |
| | | Daro_1962, VV2674, VP2431, | |
| | | YGR208W, Bxe_A2331, | |
| | | VV11730, RSc1640, blr6505, | |
| | | VF0509, CMQ250C, IL1876, | |
| | | Nwi_2345, Bcep18194_A5077, | |
| | | L0085, Z5989, NMB0981, | |
| | | SBO_4451, SSO_4538, S4691, | |
| | | SF4420, ECs5346, JW4351, | |
| | | b4388, SDY_4649, | |
| | | UTI89_C5159, c5473, | |
| | | SAK_0710, gbs0605, | |
| | | SAG0625, MJ1594, ECA0465, | |
| | | BL1792, RPB_3347, | |
| | | NMA1179, GOX1085, | |
| | | RSP_1350, Tcr_1620, SC4423, | |
| | | orf19.5838, NGO1468, | |
| | | YPTB0586, YP3740, y3738, | |
| | | YPO0442, STM4578, | |
| | | SPA4388, t4617, STY4925, | |
| | | RPA2029, VC2345, ZMO1137, | |
| | | CC2097, PPA2051, ebA6034, | |
| | | BURPS1710b_2322, | |
| | | BPSL1543, BMA1313, | |
| | | MTH1626, CV3516, | |
| | | CPS_1107, RHE_CH02794, | |
| | | AGR_C_3697, Atu2040, | |
| | | RPD_2096, Nmul_A0636, | |
| | | BTH_I2264, Msp_1096, | |
| | | BB3819, BPP3368, MK0121, | |
| | | SPO3353, BP0863, PM1657, | |
| | | SG0398, Mbur_0935, HI1033, | |
| | | NTHI1192, RPC_3257, | |
| | | Nham_2724, Noc_2504, | |
| | | mlr1449, NE0439, BR1391, | |
| | | BMEI0615, BAB1_1410, | |
| | | BruAb1_1387, plu0551, | |
| | | SMc01494, MMP0541, | |
| | | SO1223, Jann_0252, | |
| | | Bpro_2720, MS1758, amb3479, | |
| | | PSHAa0661, MA4429, | |
| | | MM1107, LIC11775, LA2145, | |
| | | Sden_1032, Mbar_A1094, | |
| | | Rfer_1329, MCA1267, | |
| | | ELI_05525, Saro_2259, | |
| | | WS2081, SPO2363, STM2197, | |
| | | NP0274A, SC2213, SPA0654, | |
| | | t0658, STY2431, PG1170, | |
| | | rrnAC2717, DDB0230054, | |
| | | CJE0330, Cj0282c, | |
| | | VNG2423G, Tmden_1665, | |
| | | HP0652, jhp0597, CMP085C, | |
| | | PAB1207, CMT542C, TK0052 | |
| SerC | phosphoserine | NCgl0794, cg0948, NCgl0794, | C. glutamicum and |
| | aminotransferase | CE0903, DIP0784, jk0425, | others |
| | | nfa6550, SAV3883, | |
| | | MAP0823c, ML2136, | |
| | | Tfu_0246, SCO4366, Mb0908c, | |
| | | MT0907, Rv0884c, | |
| | | Francci3_0082, Lxx17890, | |
| | | BL1660, PPA0483, Jann_0260, | |
| | | SPO3354, GSU3260, | |
| | | ZMO1684, Gmet_3173, | |
| | | Saro_2679, RSP_1351, | |
| | | Rru_A1104, Sde_1332, | |
| | | CG11899-PA, CPE0053, | |
| | | lp_0204, Pcar_2772, BT1153, | |
| | | DSY4684, amb3194, | |
| | | rrnAC3046, mll3876, | |
| | | NP0884A, Adeh_2622, | |
| | | BF2072, BF2018, Nham_1118, | |
| | | Moth_0019, PG1278, | |
| | | Ava_1171, RHE_CH03455, | |
| | | LMOf2365_2816, | |
| | | BT9727_3023, SMc00640, | |
| | | Mbur_0514, AGR_L_2260, | |
| | | Atu3707, all1683, BCE3285, | |
| | | CC3216, 30.t00047, lmo2825, | |
| | | Nwi_2969, BruAb1_1672, | |
| | | BR1687, BAB1_1699, SG0990, | |
| | | lin2957, BMEI0347, Tbd_0949, | |
| | | NTHI1335, BCZK2969, | |
| | | PSPPH_3666, CAC0014, | |
| | | CMT252C, GOX1446, | |
| | | RPC_4107, CV2301, | |
| | | BCI_0252, Psyr_3646, AF1417, | |
| | | MM2911, BC3249, BH1188, | |
| | | RPA4309, bll7402, | |
| | | DDB0230053, BAS3079, | |
| | | BA_3823, RPB_1314, HI1167, | |
| | | Nmul_A2190, STH3178, | |
| | | L0083, Daro_0984, Pfl_4077, | |
| | | PP1768, HD1382, 253.t00001, | |
| | | LSL_0091, ECA2594, | |
| | | PTO0371, Mbar_A1294, | |
| | | BH03780, PFL_4313, | |
| | | PSPTO1746, GBAA3321, | |
| | | BA3321, Bfl383, BQ02790, | |
| | | Mfla_1687, y2784, YPO1389, | |
| | | CNL05470, RPD_3906, | |
| | | YPTB1414, MA2304, | |
| | | SRU_2207, Daro_1231, | |
| | | RSc0903, ELI_01955, HP0736, | |
| | | Rfer_1570, PA3167, plu1619, | |
| | | MJ0959, ST0602, BG12673, | |
| | | FTT0560c, MS1573, jhp0673, | |
| | | FTL_1018, LIC10315, LA0366, | |
| | | lpp1373, RB6246, BLi01082, | |
| | | BL05093, NE0333, F26H9.5, | |
| | | DP1933, Noc_0172, | |
| | | HCH_04982, PM0837, | |
| | | PMT9312_0035, ebA907, | |
| | | Rmet_0715, Reut_A2576, | |
| | | lpg1418, PBPRA2455, VF0899, | |
| | | str1529, Adeh_2994, | |
| | | BTH_I1966, lpl1369, MM0246, | |
| | | Mbar_A2080, | |
| | | Bcep18194_A4155, Csal_2167, | |
| | | DR1350, gbs1621, | |
| | | BURPS1710b_2651, | |
| | | BPEN_394, rrnAC1999, | |
| | | Mhun_2475, Cj0326, | |
| | | Tmden_0073, BPSL2219, | |
| | | Pcryo_1434, YP1204, | |
| | | MTH1601, GK0649, Tcr_1192, | |
| | | Psyc_1036, PBPRA3292, | |
| | | stu1529, SYN_00124, | |
| | | STM0977, SPA1821, MA1816, | |
| | | SC0931, t1957, STY0977, | |
| | | TK1548, CJE0371, BMA1625, | |
| | | Dgeo_1114, XOO2388, | |
| | | SSO2597, BURPS1710b_2998, | |
| | | BMA0433, VV11425, | |
| | | UTI89_C0978, c1045, | |
| | | MK0633, HH0909, | |
| | | ACIAD2647, ABC1531, | |
| | | MCA1420, SSO_0908, S0966, | |
| | | SF0902, BPSL2519, | |
| | | WGLp486, bbp289, SBO_2193, | |
| | | AO090023000099, | |
| | | Bxe_A0976, IL1359, | |
| | | SDY_2354, ECs0990, Z1253, | |
| | | JW0890, b0907, VV21664, | |
| | | Syncc9605_0044, VP2714, | |
| | | VP1247, XC_2645, XCC1589, | |
| | | SMa1495, BTH_I1634, | |
| | | PSHAa1422, VC1159, | |
| | | cbdb_A581, STH8, VVA0476, | |
| | | SPAC1F12.07, PAB1801, | |
| | | WS0024, VV2958, TTHA0582, | |
| | | CT0070, PMM0035, | |
| | | NMA1894, VV1451, VV12813, | |
| | | VPA0235, BU312, At4g35630, | |
| | | TTC1813, RHE_PB00131, | |
| | | NMB1640, CPS_2190, | |
| | | XAC1648, TTC0213, | |
| | | Bpro_1793, Sden_0404, | |
| | | XF2326, At2g17630, PH1308, | |
| | | MMP0391, DET0600, | |
| | | Tbd_2509, VF0339, | |
| | | TTHA0173, NP2578A, | |
| | | VCA0604, Saci_0249, | |
| | | NGO1283, VC0392, SMU.1656 | |
| sdaA | serine dehydratase | NCgl1583 | C. glutamicum |
| | | NCgl0939 | |
| ThrE | threonine and serine | AAK61331 | C. glutamicum |
| | export carrier protein | | |
| SHMT | Serine | Q93PM7, BA000035, Q8FQR1, | C. glutamicum and |
| | hydroxymethyltransferase | Q6NI47, Q4JU69, Q5YQ76, | others |
| | | Q73WG1, Q4NIE8, O53441, | |
| | | P59953, Q6ADF0, Q9X794, | |
| | | Q40XZ1, Q82JI0, O86565, | |
| | | Q47MD6, Q4NM56, ORF, | |
| | | Q4NGB0, BX251412, O53615, | |
| | | P66806, Q7U2X3, Q24MM6, | |
| | | Q2ZEP1, Q65DW5, Q426V7, | |
| | | Q5KUI2, Q2RFW7, Q3A934, | |
| | | Q3CJJ0, Q8R887, Q8Y4B2, | |
| | | Q4EPI3, Q71WN9, Q67N41, | |
| | | Q927V4, P39148, Q5HE87, | |
| | | Q2YUJ1, Q9K6G4, Q7SIB6, | |
| | | Q2FF15, Q5WB66, Q4CID1, | |
| | | Q2BG18, Q40L42, Q3AN03, | |
| | | Q8YMW8, O66776, Q1YIN1, | |
| | | Q41G88, Q74CR5, Q3GAC7, | |
| | | Q3MBD8, Q2DMQ8, Q7U9J7, | |
| | | Q39V87, Q630T3, Q72XD7, | |
| | | Q2D1V8, Q6HAW9, | |
| | | AE017221, Q5SI56, Q72IH2, | |
| | | Q814V2, Q5HMB0, Q8XJ32, | |
| | | AE017225, Q81JY4, Q3WZQ2, | |
| | | CP000360, Q5NN85, | |
| | | Q3AW18, Q4L7Z4, Q3A4L9, | |
| | | Q26LA5, Q7V4U3, Q6FA66, | |
| | | Q2S9R4, Q3G5N8, Q2SFI7, | |
| | | Q3N8U1, Q6N693, Q82UP9, | |
| | | Q2JT50, Q31CS4, Q5P7P1, | |
| | | Q9HTE9, Q3KDV1, Q26XG3, | |
| | | Q3SGX5, Q5FNK4, Q2ILI1, | |
| | | S30382, Q2YD58, Q4BPZ9, | |
| | | Q2LQM6, Q5N2P9, Q376I5, | |
| | | Q46HB6, P50435, Q37NB6, | |
| | | Q7ND67, Q72CT0, Q2WMW5, | |
| | | Q2CH39, Q7VDS8, Q8U7Y5, | |
| | | Q88AD1, O85718, Q48CP3, | |
| | | Q2JI36, Q8DH33, Q7V335, | |
| | | Q214H7, Q6MLK1, Q3QXZ6, | |
| | | Q2DFI0, Q9WZH9, CP000283, | |
| | | Q37FB0, Q3N0F7, Q4ZM83, | |
| | | Q44AR5, Q8EM73, Q1WTR3, | |
| | | Q2CP12, Q3SRV3, Q3CCS2, | |
| | | Q2W4T2, Q35IU4, Q2IWS4, | |
| | | Q2CQJ5, Q4J3C4, Q49Z60, | |
| | | CT573326, Q4C6H0, Q3IZN2, | |
| | | Q607U4, P24060, Q4BQS8, | |
| | | Q41LQ8, Q7UQN2, Q2YN95, | |
| | | Q2RTB8, Q3P773, Q46RR4, | |
| | | Ser, Q47IH1, Q3JGP5, | |
| | | CT573326, Q21NP8, Q3F809, | |
| | | Q2T437, Q3F764, Q88R12, | |
| | | S15203, Q4K4P6, Q5X722, | |
| | | Q8YGG7, Q3VCK5, | |
| | | Q5WYH4, CP000271, | |
| | | Q1UEA8, Q4LV45, Q8G1F1, | |
| | | Q9I138, P77962, P34895, | |
| | | Q62DI5, Q1QMB9, Q1V9T1, | |
| | | Q2BLZ4, Q30YL7, Q8XTQ1, | |
| | | Q92QU6, Q97GV1, Q39A26, | |
| | | Q45D73, AM180252, | |
| | | Q3WQZ9, Q9KMP4, Q2KA25, | |
| | | Q4LY56, Q2S4G9, Q8D7G5, | |
| | | Q36MR4, Q28N04, Q3K5K9, | |
| | | CP000254, BA000038, | |
| | | Q4B4P5, Q2FLH5, Q7NYI8, | |
| | | Q7MEH7, Q6N622, Q2RVA2, | |
| | | Q3XRF3, Q303B4, Q7N216, | |
| | | Q47WY2, Q4UQT6, Q481S6, | |
| | | Q4BM61, Q4BA21, Q3FFQ1, | |
| | | Q3HGC4, Q87I03, Q3FB08, | |
| | | Q5LPA8, Q88UT5, Q92XS8, | |
| | | Q3QH38, Q34W82, Q39J72, | |
| | | Q8Y1G1, CP000152, | |
| | | AM236080, H97501, Q391K1, | |
| | | Q8UG75, Q21V29, Q474L3, | |
| | | Q8KC36, Q3APN5, CP000124, | |
| | | Q3BXI8, Q62I16, Q831F9, | |
| | | Q1QE01, Q3CX04, Q2NZ83, | |
| | | Q8PPE3, CP000352, Q3S0V7, | |
| | | Q2AFR6, Q8TK94, CP000086, | |
| | | Q2BI80, Q2G646, Q3J9K8, | |
| | | Q47XG4, Q2SYS4, Q1YWG2, | |
| | | Q73GC3, Q44LK7, Q33Y20, | |
| | | Q2NS25, Q2CGY4, Q5GTS7, | |
| | | Q36D93, AE008384, Q8PZQ0, | |
| | | Q9HVI7, Q983B6, CP000270, | |
| | | Q3R0R3, Q2Z5R9, Q1VX33, | |
| | | Q4ZNH2, Q4FUZ8, Q72PY2, | |
| | | Q48DU7, Q3VPD3, Q6LHN7, | |
| | | AE009442, Q87AS2, Q3B2I7, | |
| | | Q87WC1, Q7WFD2, Q4K5R9, | |
| | | Q1R8I4, P0A826, CT573326, | |
| | | Q3WKF8, Q2ZQD2, Q8EBN8, | |
| | | Q8XA55, Q3ZZG3, Q2J6M3, | |
| | | Q2DUP7, Q9XAZ1, Q3K6J0, | |
| | | Q3Q439, Q9A8J6, Q7W400, | |
| | | Q3YZ04, Q32D21, Q5F8C0, | |
| | | Q4AMK6, Q6D246, Q3R828, | |
| | | E82743, Q9PET2, Q3P6F8, | |
| | | Q9XAY7, Q3NK51, Q3Z9B9, | |
| | | Q6G3L3, Q88Q27, Q1ZIE9, | |
| | | Q31FS6, P56990, Q9XB01, | |
| | | Q3DHL3, Q6LU17, Q7W1I6, | |
| | | H82258, Q9KTG1, Q8E5C6, | |
| | | Q57LF7, Q3IRX5, Q3K122, | |
| | | Q7WPH6, AP008231, | |
| | | Q1YU48, Q3D8P3, Q8DPZ0, | |
| | | Q97R16, Q46A52, Q6F211, | |
| | | Q1PZE1, Q8L372, B48427, | |
| | | Q2KV15, Q1RGX5, Q43K52, | |
| | | Q3VUL2, Q3II23, Q1ZPS2, | |
| | | Q2NAR9, Q8DU67, Q9CHW7, | |
| | | Q6CZV5, Q3XBK9, H84295, | |
| | | Q4FLT4, Q1UZA1, Q8DFC9, | |
| | | Q74LC1, Q488N6, Q2C6B3, | |
| | | Q65T08, Q1Z7P1, F75567, | |
| | | Q9HPY5, Q9RYB2, Q1V311, | |
| | | Q87RR2, Q3GI80, Q6G009, | |
| | | Q8ZCR1, Q5QXT4, Q5V3D7, | |
| | | Q2ST43, Q5E706, Q8Z2Z9, | |
| | | Q1XXG3, Q5PBM8, Q6MS85, | |
| | | Q3EFW1, Q7QM11, Q2BUE3, | |
| | | Q48TK6, Q5FMC0, | |
| | | BA000034, Q1U7W2, Q8P122, | |
| | | Q8K7H8, Q99ZP1, Q5M0B4, | |
| | | Q5XC65, Q83BT3, Q2GEI3, | |
| | | Q4QM19, CP000262, Q84FT0, | |
| | | Q5M4W1, CP000260, | |
| | | Q1QU94, Q4HIU1, P43844, | |
| | | Q40IP4, Q5NFJ3, Q2A498, | |
| | | Q92GH7, Q2GLH3, O08370, | |
| | | AY871942, Q68W07, | |
| | | Q4UK96, Q4HBL3, Q30P60, | |
| | | Q26C95, Q38WJ7, Q3YRD1, | |
| | | P59432, Q7P9P7, JQ1016, | |
| | | P57830, P24531, P34894, | |
| | | Q5HW65, Q2X6F1, Q2JFD4, | |
| | | Q2NIT8, Q30R29, CP000238, | |
| | | Q6YR37, Q8A9S7, Q5LD58, | |
| | | Q5FG30, O51547, Q4HNY8, | |
| | | Q4HFT7, Q8K9P2, P57376, | |
| | | Q6AM21, Q3W273, Q660S1, | |
| | | P78011, Q6KHH3, Q4A6A3, | |
| | | Q98QM2, Q492D5, Q2DZD3, | |
| | | Q89HS7, Q7MAR0, | |
| | | Q7MXW0, Q8D253, Q8EWD1, | |
| | | Q7NBH8, Q7VFL1, Q4QTL5, | |
| | | P56089, Q3W5W4, Q601P7, | |
| | | Q2E435, Q7VRR4, Q4A8E1, | |
| | | P47634, Q4AAB2, Q9ZMP7, | |
| | | Q82J74, Q1VNH3, Q50LF3, | |
| | | Q3WZI8, Q9K4E0, Q8KJG9, | |
| | | Q98A81, I40886, P50434, | |
| | | Q9W457, Q30K91, Q30K95, | |
| | | Q30K92, Q30K98, Q30K94, | |
| | | Q30K93, Q5H888, Q29H49, | |
| | | Q1UKA7, Q3KLR8, Q6U9U4, | |
| | | Q56F03, Q268J4, Q275S8, | |
| | | Q4I358, Q758F0, Q6CLQ5, | |
| | | CH476726, Q94JQ3, T05362, | |
| | | Q5L6P4, AJ438778, Q5B0U5, | |
| | | S24342, P07511, Q7SXN1, | |
| | | Q2KIP4, Q5E9P9, S65688 | |
| MetF | Methylene | Q8NNM2, AX374883, | C. glutamicum and |
| | tetrahydrofolate reductase | AX064391, Q8FNS7, | others |
| | | Q6NGB6, Q47R29, Q938W5, | |
| | | Q2DXH2, Q82AF8, Q3W2U6, | |
| | | O54235, T34973, Q3H080, | |
| | | Q2JD76, O67422, Q2ILB5, | |
| | | Q5SLG6, Q3VN87, AJ416377, | |
| | | Q40UK0, Q6AMT4, Q8KCP5, | |
| | | Q3A3T2, Q36NE9, Q3GGT1, | |
| | | Q3B375, Q3ARK5, Q4CIZ2, | |
| | | Q2CBP7, Q40RF5, Q44MN6, | |
| | | Q3VVL1, Q2RU65, Q2L158, | |
| | | Q72DD2, Q2N880, Q7VUM0, | |
| | | Q4AKX3, CQ795554, Q43FI9, | |
| | | Q3G6N2, P11003, X07689, | |
| | | Q10258, Q83SU8, AB0937, | |
| | | Q4UQY4, P71319, Q7MYD0, | |
| | | Q2NQY8, Q4X140, Q87L52, | |
| | | Q93ER8, B86085, P0AEZ1, | |
| | | Q3VG92, Q8DCN4, Q7MH66, | |
| | | Q1R3X1, Q5I598, Q6J6A1, | |
| | | Q65UG6, Q60CG9, Q5NLN9, | |
| | | Q1V6F7, Q8PP97, Q47JN9, | |
| | | Q3J4G1, C87514, Q8Y389, | |
| | | Q3P3Q2, Q21NJ7, Q87V72, | |
| | | Q4HZN6, Q3QI84, Q1YM37, | |
| | | Q26NU6, Q4J1U0, Q36HT8, | |
| | | Q2D3T4, Q2DGG5, AJ237672, | |
| | | BC053509, CS287591, | |
| | | CS287593, Q3K5D6, Q59GJ6, | |
| | | Q5SNW6, C82045, F81880, | |
| | | P42898, P45208, Q1QR87, | |
| | | Q30YC4, Q1YZZ8, Q2Y5Z3, | |
| | | CQ795570, AM236080, | |
| | | Q6LVH3, Q3QYB9, Q3RDV3, | |
| | | CP000270, Q5F862, D81140, | |
| | | Q60HE5, Q88D51, Q87EA5, | |
| | | Q1R0K0, Q3NPK8, Q4FT38, | |
| | | Q2ZW39, Q3F2W2, S46454, | |
| | | CT573326, Q8EA55, Q1QAS5, | |
| | | Q476T6, Q2G3D0, Q2X8T6,, | |
| | | Q7UNJ7, Q3RB96, Q33Z96, | |
| | | Q4NA26, Q3SFY6, Q44YV3, | |
| | | Q2K697, CQ795568, Q3Q3H0, | |
| | | Q2STU2 | |

In addition to increasing the amount of serine available to the microorganism, either by providing an increased amount of serine in the medium or by genetic modification of the microorganism with respect to proteins involved in serine metabolism and transport, the microorganism may also be genetically modified to express one or more enzymes involved in methionine synthesis. This will lead to an even higher increase in methionine yield or the efficiency of methionine synthesis. These enzymes may be selected from the group consisting of aspartokinase (lysC), homoserine dehydrogenase (hom), homoserine-O-acetyl transferase (MetA), O-acetyl homoserine sulfhydrolase (MetZ), cob(I)alamin dependent methionine synthase I (MetH) and cob(I)alamin independent methionine synthase II (MetE). Nucleic acid sequences coding for these proteins may be obtained from the respective database, e.g. at NCBI (http://www.ncbi.nlm.nih.gov/), EMBL (http://www.embl.org), Expasy (http://www.expasy.org/), KEGG (http://www.genome.ad.jp/kegg/kegg.html) etc. Specific examples for enzymes involved in methionine biosynthesis are given in Table 2.

TABLE 2

| Name | Enzyme | Gene bank accession number | Organism |
|---|---|---|---|
| MetA | homoserine O- | Cg10652, cg0754, CE0678, | C. glutamicum and |
| | acetyltransferase | DIP0623, jk1695, nfa9220, | others |
| | | MAP3458, ML0682, Mb3373, | |
| | | Rv3341, MT3444, Tfu_2822, | |
| | | Lxx18950, CT0605, blr1399, | |
| | | STH1685, CC0525, ZMO0225, | |
| | | RPA4437, MA2714, GOX0203, | |
| | | mlr3538, DP1243, LIC11853, | |
| | | LA2061, BPP4083, BP0047, | |
| | | BB4554, GSU2462, BMA3246, | |
| | | BPSL0197, SAR11_0217, | |
| | | ebA2806, VNG2420G, | |
| | | Daro_0130, CV0786, AFR682C, | |
| | | HI1263, RB8222, NGO0933, | |
| | | LMOf2365_0623, RSc0027, | |
| | | lmo0594, NTHI1901, lin0603, | |
| | | YNL277W, NMB0940, MS0924, | |
| | | orf19.2618, rrnAC3064, PD1484, | |
| | | NMA1136, PM0866, TTC0407, | |
| | | TTHA0759, XF2465, NE2186, | |
| | | PSPPH_0465, PSPTO5049, | |
| | | SPBC56F2.11, Psyr_0474, | |
| | | XC_1889, XCC2228, PP5097, | |
| | | PFL_5842, ACIAD0529, | |
| | | XOO2093, PA0390, XAC2332, | |
| | | CNE02740, WS1893, Psyc_0375, | |
| | | DR0872, IL2157, BA4983, | |
| | | BAS4629, GBAA4983, BC4730, | |
| | | BCZK4482, BT9727_4463, | |
| | | BCE4873, SAR0012, | |
| | | SACOL0012, MW0012, | |
| | | SAS0012, SA0011, SAV0012, | |
| | | SH0011, SE0011, SERP2541, | |
| | | MTH1820, gll2500, BA_5402 | |
| MetE | cob(I)alamin | Cgl1507, cg1290, CE1209, | C. glutamicum |
| | independent | jk0234, Mb1164c, MT1165, | and others |
| | methionine | Rv1133c, MAP2661, ML0961, | |
| | synthase II | SCO0985, PM0420, SAV2046, | |
| | | CMJ234C, NE1436, PD1308, | |
| | | CC0482, XF2272, RSp0676, | |
| | | HI1702, CV3604, NGO0928, | |
| | | MCA2260, At5g17920, | |
| | | ZMO1000, RPA2397, BB2079, | |
| | | BPP2636, BP2543, NMA1140, | |
| | | NMB0944, mll6123, BPSL2545, | |
| | | BMA0467, SPAC9.09, YPO3788, | |
| | | YP3261, y0442, YPTB0248, | |
| | | SF3907, S3848, PSPTO4179, | |
| | | SC3864, CBU2048, STM3965, | |
| | | JW3805, b3829, DVU3371, | |
| | | Z5351, ECs4759, t3332, | |
| | | STY3594, SPA3806, WS0269, | |
| | | blr2068, ECA0181, PFL_2404, | |
| | | plu4420, nfa52280, CNK02310, | |
| | | PA1927, PBPRA1379, VV12219, | |
| | | VF1721, VC1704, VV2135, | |
| | | VP1974, bbp031, BL0798, | |
| | | SO0818, BU030, BUsg031, | |
| | | SP0585, HH0852, spr0514, | |
| | | orf19.2551, ABR212C, str0785, | |
| | | stu0785, lmo1681, YER091C, | |
| | | BH0438, LMOf2365_1705, | |
| | | Bfl625, lp_1375, BLi01422, | |
| | | BL03738, lin1789, SMU.873, | |
| | | DDB0230069, BT9727_3744, | |
| | | ABC1449, tlr1090, BA4218, | |
| | | GBAA4218, BAS3912, | |
| | | BCE4053, BC4003, CJE1335, | |
| | | L0100, BA_4680, Cj1201, | |
| | | SA0344, SAV0356, SACOL0428, | |
| | | SERP0034, MW0332, SAR0353, | |
| | | SE2382, SAS0332, TM1286, | |
| | | BCZK3760, SH2638, BG12616, | |
| | | SAG2049, gbs2005, aq_1710, | |
| | | TW610, TWT162, APE2048, | |
| | | SSO0407, ST0385, Saci_0828, | |
| | | rrnAC0254, PF1269, TK1446, | |
| | | PAB0608, PH1089, PAE3655, | |
| | | Ta0977, MTH775, XC_0330, | |
| | | XCC0318, Psyc_0846, GOX2206, | |
| | | TVN1123, ACIAD3523, | |
| | | AGR_L_2018, Atu3823, | |
| | | PTO0186, XAC0336, Psyr_2855, | |
| | | MJ1473, PP2698, XOO4333, | |
| | | CPS_1151, MK0667, | |
| | | PSPPH_3910, MMP0401 | |
| MetH | cob(I)alamin | Cgl1139, cg1701, CE1637, | C. glutamicum |
| | dependent | DIP1259, nfa31930, Rv2124c, | and others |
| | methionine synthase I | Mb2148c, ML1307, SCO1657, | |
| | | Tfu_1825, SAV6667, MT2183, | |
| | | GOX2074, tll1027, syc0184_c, | |
| | | alr0308, slr0212, gll0477, | |
| | | SYNW1238, TTC0253, | |
| | | TTHA0618, PMT0729, Pro0959, | |
| | | PMN2A_0333, PMM0877, | |
| | | WS1234, BH1630, GK0716, | |
| | | BCE4332, ABC1869, BC4250, | |
| | | BCZK4005, BT9727_3995, | |
| | | BA_4925, GBAA4478, BA4478, | |
| | | BAS4156, BLi01192, BL01308, | |
| | | MAP1859c, BruAb1_0184, | |
| | | BMEI1759, BR0188, SMc03112, | |
| | | MCA1545, AGR_C_3907, | |
| | | Atu2155, DR0966, RB9857, | |
| | | ebA3184, VC0390, RPA3702, | |
| | | VV11423, VV2960, VP2717, | |
| | | NE1623, VF0337, LIC20085, | |
| | | LB108, YPTB3653, YPO3722, | |
| | | y0020, YP3084, CV0203, | |
| | | SPA4026, MS1009, SC4067, | |
| | | SO1030, DP2202, STM4188, | |
| | | STY4405, t4115, PP2375, | |
| | | PFL_3662, Z5610, ECs4937, | |
| | | c4976, JW3979, b4019, SF4085, | |
| | | S3645, BB4456, BPP3983, | |
| | | BP3594, bll1418, CPS_1101, | |
| | | Psyr_2464, PSPTO2732, | |
| | | R03D7.1, PSPPH_2620, | |
| | | PBPRA3294, Daro_0046, | |
| | | PA1843, ECA3987, CT1857, | |
| | | CAC0578, ACIAD1045, | |
| | | Psyc_0403, 4548, DDB0230138, | |
| | | BF3039, BF3199, BT0180, | |
| | | 238505, GSU2921, STH2500, | |
| | | XC_2725, XCC1511, XOO2073, | |
| | | TTE1803, RSc0294, XAC1559, | |
| | | BPSL0385, DVU1585, | |
| | | CTC01806, CC2137, TM0268, | |
| | | ZMO1745, FN0163, BG13115, | |
| | | lin1786, SAG2048, gbs2004, | |
| | | LMOf2365_1702, lmo1678, | |
| | | SE2381, SERP0035, MW0333, | |
| | | SAS0333, SMU.874, SA0345, | |
| | | SAV0357, SACOL0429, | |
| | | SAR0354, SH2637 | |
| MetZ | O- | NCgl0625, cg0755, CE0679, | C. glutamicum |
| | acetylhomoserine | DIP0630, jk1694, MAP3457, | and others |
| | sulfhydrolase | Mb3372, MT3443, Rv3340, | |
| | | nfa35960, Lxx18930, Tfu_2823, | |
| | | CAC2783, GK0284, BH2603, | |
| | | lmo0595, lin0604, | |
| | | LMOf2365_0624, ABC0432, | |
| | | TTE2151, BT2387, STH2782, | |
| | | str0987, stu0987, BF1406, | |
| | | SH0593, BF1342, lp_2536, | |
| | | L75975, OB3048, BL0933, | |
| | | LIC11852, LA2062, BMAA1890, | |
| | | BPSS0190, SMU.1173, BB1055, | |
| | | PP2528, PA5025, PBPRB1415, | |
| | | GSU1183, RPA2763, WS1015, | |
| | | TM0882, VP0629, BruAb1_0807, | |
| | | BMEI1166, BR0793, CPS_2546, | |
| | | XC_1090, XCC3068, plu3517, | |
| | | PMT0875, SYNW0851, Pro0800, | |
| | | CT0604, NE1697, RB8221, | |
| | | bll1235, syc1143_c, ACIAD3382, | |
| | | ebA6307, RSc1562, Daro_2851, | |
| | | DP2506, DR0873, MA2715, | |
| | | PMM0642, PMN2A_0083, | |
| | | IL2014, SPO1431, ECA0820, | |
| | | AGR_C_2311, Atu1251, | |
| | | mlr8465, SMc01809, CV1934, | |
| | | SPBC428.11, PM0738, SO1095, | |
| | | SAR11_1030, PFL_0498, | |
| | | CTC01153, BA_0514, BCE5535, | |
| | | BAS5258, GBAA5656, BA5656, | |
| | | BCZK5104, TTHA0760, | |
| | | TTC0408, BC5406, | |
| | | BT9727_5087, HH0636, | |
| | | YLR303W, ADL031W, CJE1895, | |
| | | spr1095, rrnAC2716, orf19.5645, | |
| | | Cj1727c, VNG2421G, | |
| | | PSPPH_1663, XOO1390, | |
| | | Psyr_1669, PSPTO3810, | |
| | | MCA2488, TDE2200, FN1419, | |
| | | PG0343, Psyc_0792, MS1347, | |
| | | CC3168, Bd3795, MM3085, | |
| | | 389.t00003, NMB1609, | |
| | | SAV3305, NMA1808, GOX1671, | |
| | | APE1226, XAC3602, NGO1149, | |
| | | ZMO0676, SCO4958, lpl0921, | |
| | | lpg0890, lpp0951, EF0290, | |
| | | BPP2532, CBU2025, BP3528, | |
| | | BLi02853, BL02018, BG12291, | |
| | | CG5345-PA, HP0106, ML0275, | |
| | | jhp0098, At3g57050, 107869, | |
| | | HI0086, NTHI0100, | |
| | | SpyM3_0133, SPs0136, | |
| | | spyM18_0170, M6_Spy0192, | |
| | | SE2323, SERP0095, SPy0172, | |
| | | PAB0605, DDB0191318, | |
| | | ST0506, F22B8.6, PTO1102, | |
| | | CPE0176, PD1812, XF0864, | |
| | | SAR0460, SACOL0503, SA0419, | |
| | | Ta0080, PF1266, MW0415, | |
| | | SAS0418, SSO2368, PAE2420, | |
| | | TK1449, 1491, TVN0174, | |
| | | PH1093, VF2267, Saci_0971, | |
| | | VV11364, CMT389C, VV3008 | |
| lysC | aspartokinase | NCgl0247, CE0220, DIP0277, | |
| | | jk1998, nfa3180, Mb3736c, | |
| | | MT3812, Rv3709c, ML2323, | |
| | | MAP0311c, Tfu_0043, | |
| | | Francci3_0262, SCO3615, | |
| | | SAV4559, Lxx03450, PPA2148, | |
| | | CHY_1909, MCA0390, | |
| | | cbdb_A1731, TWT708, TW725, | |
| | | Gmet_1880, DET1633, GSU1799, | |
| | | Moth_1304, Tcr_1589, | |
| | | Mfla_0567, HCH_05208, | |
| | | PSPPH_3511, Psyr_3555, | |
| | | PSPTO1843, CV1018, STH1686, | |
| | | NMA1701, Tbd_0969, | |
| | | NMB1498, Pcar_1006, | |
| | | Daro_2515, Csal_0626, | |
| | | Tmden_1650, PA0904, PP4473, | |
| | | Sde_1300, HH0618, NGO0956, | |
| | | ACIAD1252, PFL_4505, ebA637, | |
| | | Noc_0927, WS1729, Pcryo_1639, | |
| | | Psyc_1461, Pfl_4274, LIC12909, | |
| | | LA0693, Rru_A0743, NE2132, | |
| | | RB8926, Cj0582, Nmul_A1941, | |
| | | SYN_02781, TTHA0534, | |
| | | CJE0685, BURPS1710b_2677, | |
| | | BPSL2239, BMA1652, RSc1171, | |
| | | TTC0166, RPA0604, | |
| | | BTH_I1945, Bpro_2860, | |
| | | Rmet_1089, Reut_A1126, | |
| | | RPD_0099, Bxe_A1630, | |
| | | Bcep18194_A5380, aq_1152, | |
| | | RPB_0077, Rfer_1353, | |
| | | RPC_0514, BH3096, BLi02996, | |
| | | BL00324, amb1612, tlr1833, | |
| | | jhp1150, blr0216, Dde_2048, | |
| | | BB1739, BPP2287, BP1913, | |
| | | DVU1913, Nwi_0379, ZMO1653, | |
| | | Jann_3191, HP1229, Saro_3304, | |
| | | Nham_0472, CBU_1051, slr0657, | |
| | | SPO3035, Synpcc7942_1001, | |
| | | BG10350, BruAb1_1850, | |
| | | BAB1_1874, BMEI0189, | |
| | | BT9727_1658, syc0544_d, | |
| | | BR1871, gll1774, BC1748, | |
| | | mll3437, BCE1883, ELI_14545, | |
| | | RSP_1849, BCZK1623, | |
| | | BAS1676, BA_2315, | |
| | | GBAA1811, BA1811, Ava_3642, | |
| | | alr3644, PSHAa0533, | |
| | | AGR_L_1357, Atu4172, lin1198, | |
| | | BH04030, PMT9312_1740, | |
| | | SMc02438, CYA_1747, | |
| | | RHE_CH03758, lmo1235, | |
| | | LMOf2365_1244, PMN2A_1246, | |
| | | CC0843, Pro1808, BQ03060, | |
| | | PMT0073, Syncc9902_0068, | |
| | | GOX0037, CYB_0217 | |
| Hom | homoserine | cg1337, NCgl1136, CE1289, | |
| | dehydrogenase | DIP1036, jk1352, nfa10490, | |
| | | SAV2918, Mb1326, MT1333, | |
| | | Rv1294, SCO5354, MAP2468c, | |
| | | ML1129, Francci3_3725, | |
| | | Tfu_2424, Lxx06870, PPA1258, | |
| | | Moth_1307, BL1274, CHY_1912, | |
| | | DSY1363, GK2964, CAC0998, | |
| | | BLi03414, BL02137, BC5404, | |
| | | STH2739, BCZK5102, | |
| | | BT9727_5085, Gmet_1629, | |
| | | BCE5533, BB1926, BP2784, | |
| | | CTC02355, BG10460, BPP2479, | |
| | | BAS5256, BA_0512, | |
| | | GBAA5654, BA5654, | |
| | | Synpcc7942_2090, syc2003_c, | |
| | | Adeh_1638, CYA_1100, | |
| | | Pcar_1451, Mfla_1048, | |
| | | Mfla_0904, TW329, TWT439, | |
| | | BH3422, all4120, Daro_2386, | |
| | | gll4295, ebA4952, Ava_0783, | |
| | | Syncc9605_1957, LSL_1519, | |
| | | OB0466, lmo2547, PMT1143, | |
| | | Bpro_2190, SYNW0711, | |
| | | LMOf2365_2520, lin2691, | |
| | | sll0455, CV0996, RSc1327, | |
| | | PMT9312_1062, ABC2942, | |
| | | Bcep18194_A5155, | |
| | | BURPS1710b_2396, BPSL1477, | |
| | | BMA1385, NMA1395, | |
| | | NMB1228, tll0277, | |
| | | Syncc9902_0704, GSU1693, | |
| | | Bxe_A2381, MCA0597, | |
| | | NGO0779, CYB_1425, | |
| | | BTH_I2198, BMEI0725, | |
| | | Rmet_1966, Rfer_1912, | |
| | | SMc00293, BruAb1_1275, | |
| | | BAB1_1293, SYN_00890, | |
| | | Reut_A1993, RHE_CH01878, | |
| | | BR1274, aq_1812, TTE2620, | |
| | | ACIAD0264, PFL_1103, stu0469, | |
| | | str0469, Pfl_1027, Psyr_1290, | |
| | | PMN2A_0702, MTH1232, | |
| | | Csal_3010, AGR_C_2919, | |
| | | Atu1588, PSPPH_1360, PP1470, | |
| | | NE2369, PSPTO1480, Tcr_1251, | |
| | | BC1964, Nmul_A1551, | |
| | | Saro_0019, mll0934, WS0450, | |
| | | spr1219, SP1361, Noc_2454, | |
| | | BT9727_1799, BCZK1782, | |
| | | BCE2051, Tbd_0843, PA3736, | |
| | | DET1206, amb3728, Rru_A2410, | |
| | | LIC10571, LA3638, SMU.965, | |
| | | BAS1825, BA_2468, | |
| | | GBAA1968, BA1968, | |
| | | cbdb_A1123, GOX1517, | |
| | | PMM1051, HCH_01779, | |
| | | RB8510, DVU0890, Pro1150, | |
| | | Nham_2309, Tmden_1904, | |
| | | Sde_1209, Psyc_0253, | |
| | | ELI_13775, RSP_0403, L0090, | |
| | | Dde_2731, Pcryo_0279, | |
| | | Nwi_1647, lp_0571, BH10030, | |
| | | SPO1734, Jann_2998, blr4362, | |
| | | RPA2504, EF2422, DP1732, | |
| | | LBA1212, RPD_2495, | |
| | | RPC_2816, CC1383, RPB_2966, | |
| | | CJE0145, Cj0149c, | |
| | | Acid345_1481, ZMO0483, | |
| | | Bpro_5333, SAK_1205, gbs1187, | |
| | | jhp0761, SH1579, SAG1120, | |
| | | HP0822, SE1009, SERP0897, | |
| | | SAOUHSC_01320, | |
| | | SAUSA300_1226, SAB1186, | |
| | | SACOL1362, SAS1268, | |
| | | SAR1338, MW1215, SAV1328, | |
| | | SA1164, HH1750, SSP1438, | |
| | | lp_2535, TTE2152, SAR11_1025, | |
| | | DR1278, PFL_3809, Dgeo_0610, | |
| | | Mhun_2292, DSY3981, PP0664, | |
| | | MA2572, ABC1578, | |
| | | Mbar_A1898, TTHA0489, | |
| | | TTC0115, MM2713, Mbur_1087, | |
| | | BH1737, AF0935, MK1554, | |
| | | MTH417, VNG2650G, | |
| | | Msp_0487, ABC0023, | |
| | | rrnAC2408, TK1627, TM0547, | |
| | | MJ1602, NP0302A, BH1253, | |
| | | MMP1702, BCE2626, | |
| | | LmjF07.0260, BCZK2354, | |
| | | BT9727_2388, BAS2433, | |
| | | BA_3119, GBAA2608, BA2608, | |
| | | BC2548, Acid345_4165, | |
| | | CTC00886, ST1519, Saci_1636, | |
| | | APE1144, SSO0657, PF1104, | |
| | | Adeh_3931, PAB0610, PH1075, | |
| | | Cag_0142, PAE2868, YJR139C, | |
| | | XOO1820, Plut_1983, XAC3038, | |
| | | Adeh_1400, XCV3175, PTO1417, | |
| | | SCO0420, SRU_0482, XC_1253, | |
| | | XCC2855, SO4055, CT2030, | |
| | | SPBC776.03, AO090003000721, | |
| | | TVN0385, ABL080W, | |
| | | AO090009000136, CPS_0456, | |
| | | HI0089, orf19.2951, Sden_0616, | |
| | | UTI89_C4525, Afu3g11640, | |
| | | MS1703, SBO_3960, SSO_4114, | |
| | | STM4101, SC3992, t3517, | |
| | | STY3768, c4893, ECs4869, | |
| | | Z5495, JW3911, b3940, | |
| | | AN2882.2, ECA4251, CMN129C, | |
| | | NTHI0167, plu4755, ECA3891, | |
| | | YPTB0602, YP3723, y3718, | |
| | | YPO0459, PM0113, S3729, | |
| | | SF4018, SPA3944, Mfla_1298, | |
| | | PSHAa2379, PBPRA0262, | |
| | | XOO2242, STM0002, SC0002, | |
| | | SPA0002, t0002, STY0002, | |
| | | c0003, SRU_0691, XCC1800, | |
| | | PD1273, BPEN_115, SDY_3775, | |
| | | VC2684, SDY_0002, SBO_0001, | |
| | | YPTB0106, YP0118, y0303, | |
| | | YPO0116, UTI89_C0002, | |
| | | ECs0002, Z0002, JW0001, b0002, | |
| | | VV3007, VV11365, XC_2389, | |
| | | VP2764, XF2225, SSO_0002, | |
| | | S0002, SF0002 | |

Another way of increasing the methionine synthesis in addition to providing an increased amount of serine available for the metabolism of the microorganism is to decrease the content and/or the biological activity of one or more transcriptional regulator proteins which are involved in the repression of genes involved in methionine synthesis. One example of such a repressor gene is disclosed in WO 02/097096 and is called McbR or MetD. The attenuation of this transcriptional regulator improves the production of L-methionine in coryneform bacteria.

An increase of the amount and/or activity of the enzymes of Table 1 and/or 2 is achieved by introducing nucleic acids encoding the enzymes of Table 1 and/or 2 into the organism, preferably C. glutamicum or E. coli.

In principle, proteins of different organisms having the enzymatic activity of the proteins listed in Table 1 and 2 can be used, if increasing the amount and/or activity is envisaged. When nucleic acid sequences from eukaryotic sources containing introns should be expressed in a cell that is not capable or cannot be made capable of splicing the corresponding mRNAs already processed nucleic acid sequences like the corresponding cDNAs are to be used. All nucleic acids mentioned in the description can be, e.g., an RNA, DNA or cDNA sequence.

In order to produce an organism that is more efficient in methionine synthesis, changing the amount and/or activity of an enzyme is not limited to the enzymes listed in Table 1 and 2. Any enzyme that is homologous to the enzymes of Table 1 and 2 and carries out the same function in another organism may be perfectly suited to modulate the amount and/or activity in order to influence the metabolic flux by way of over-expression. The definitions for homology and identity are given below.

In one process according to the present invention for preparing L-methionine by cultivating a microorganism, one or more nucleic acid sequences coding for one of the above-mentioned functional or non-functional, feedback-regulated or feedback-independent enzymes is transferred to a microorganism such as C. glutamicum or E. coli., respectively. This transfer leads to an increase of the expression of the enzyme and correspondingly to a higher metabolic flux through the desired reaction pathway.

According to the present invention, increasing the amount and/or the activity of a protein in a specific organism typically comprises the following steps:

a) production of a vector comprising the following nucleic acid sequences, preferably DNA sequences, in 5′-3′-orientation:
a promoter sequence functional in the organism;
operatively linked thereto a DNA sequence coding for a protein of Table 1 or 2 or functional equivalent parts thereof;
a termination sequence functional in the organism;
   b) transfer of the vector from step a) to the organism and, optionally, integration into the respective genomes.

If more than one nucleic acid sequence encoding proteins involved in serine metabolism and transport and/or methionine synthesis is to be introduced into the microorganism, the different nucleic acid sequences may be located on the same vector or on different vectors. If they are located on different vectors, these can be introduced into the microorganism simultaneously or subsequently.

Functionally equivalent parts of enzymes within the scope of the present invention are intended to mean fragments of nucleic acid sequences coding for enzymes of Table 1 or 2, the expression of which still leads to proteins having the enzymatic activity of the respective full length protein. The enzymatic activity can be determined by methods described in the prior art (see, e.g., Cho et al. (2001) Proc. Natl. Acad. Sci. USA 98: 8525-8530 for an assay of SerB activity; Peters-Wendisch et al. (2002) Appl. Microbiol. Biotechnol. 60: 437-441 for an assay of SerA activity).

According to the present invention, non-functional enzymes have the same nucleic acid sequences and amino acid sequences, respectively, as functional enzymes and functionally equivalent parts thereof, respectively, but have, at some positions, point mutations, insertions or deletions of nucleotides or amino acids, which have the effect that the non-functional enzymes are not, or only to a very limited extent, capable of catalyzing the respective reaction. These non-functional enzymes differ from enzymes that are still capable of catalyzing the respective reaction, but are not feed-back regulated anymore. Non-functional enzymes also comprise enzymes bearing point mutations, insertions, or deletions at the nucleic acid sequence level or amino acid sequence level which are not, or less, capable of interacting with physiological binding partners of the enzymes, such as their substrates. Non-functional mutants cannot catalyze a reaction which the wild-type enzyme, from which the mutant is derived, is capable to catalyze.

According to the present invention, the term “non-functional enzyme” does not comprise such genes or proteins having no essential sequence homology to the respective functional enzymes at the amino acid level and nucleic acid level, respectively. Proteins unable to catalyze the respective reactions and having no essential sequence homology with the respective enzyme are therefore, by definition, not meant by the term “non-functional enzyme” of the present invention. Non-functional enzymes are, within the scope of the present invention, also referred to as inactivated or inactive enzymes.

Therefore, non-functional enzymes of Table 1 and 2 according to the present invention bearing the above-mentioned point mutations, insertions, and/or deletions are characterized by an essential sequence homology to the wild type enzymes of Table 1 and 2 according to the present invention or functionally equivalent parts thereof.

Of course, the invention can also be performed with nucleic acid molecules sharing substantial sequence homology with the nucleic acid sequences coding for the proteins of Table 1 or 2 and which code for functionally equivalent proteins.

According to the present invention, a substantial sequence homology is generally understood to indicate that the nucleic acid sequence or the amino acid sequence, respectively, of a DNA molecule or a protein, respectively, is at least 40%, preferably at least 50%, further preferred at least 60%, also preferably at least 70%, particularly preferred at least 90%, in particular preferred at least 95% and most preferably at least 98% identical to the nucleic acid sequences or the amino acid sequences, respectively, of the proteins of Table 1 or 2 or functionally equivalent parts thereof.

Identity of two proteins is understood to be the identity of the amino acids over the respective entire length of the protein, in particular the identity calculated by comparison with the assistance of the Lasergene software by DNA Star, Inc., Madison, Wis. (USA) applying the CLUSTAL method (Higgins et al. (1989) Comput. Appl. Biosci. 5(2): 151).

Identity of DNA sequences is to be understood correspondingly. Nucleic acid molecules are identical, if they have identical nucleotides in identical 5′-3′-order.

The above-mentioned method can be used for increasing the expression of DNA sequences coding for functional or non-functional, feedback-regulated or feedback-independent enzymes of Table 1 and/or 2 or functionally equivalent parts thereof. The use of the above-mentioned vectors comprising regulatory sequences, such as promoter and termination sequences, is known to the person skilled in the art. Furthermore, the person skilled in the art knows how a vector from step a) can be transferred to organisms such as C. glutamicum or E. coli and which properties a vector must have to be integrated into their genomes.

If the content of a specific protein in an organism such as, e.g., C. glutamicum is to be increased by transferring a nucleic acid coding for that protein from another organism, like e.g. E. coli, it is advisable to back-translate the amino acid sequence encoded by the nucleic acid sequence e.g. from E. coli according to the genetic code into a nucleic acid sequence comprising mainly those codons, which are used more often in C. glutamicum due to the organism-specific codon usage. The codon usage can be determined by means of computer evaluations of other known genes of the relevant organisms.

According to the present invention, an increase of the gene expression and of the activity, respectively, of a nucleic acid encoding a protein of Table 1 or 2 is also understood to be the manipulation of the expression of the respective endogenous proteins of an organism, in particular of C. glutamicum or E. coli. This can be achieved, e.g., by altering the promoter DNA sequence for genes encoding these proteins. Such an alteration, which causes an altered, preferably increased, expression rate of these enzymes can be achieved by deletion or insertion of DNA sequences.

Furthermore, an altered and increased expression, respectively, of an endogenous gene can be achieved by a regulatory protein, which does not occur in the transformed organism, and which interacts with the promoter of these genes. Such a regulator can be a chimeric protein consisting of a DNA binding domain and a transcriptional activator domain, as e.g. described in WO 96/06166.

A further possibility for increasing the expression of endogenous genes is to up-regulate transcription factors involved in the transcription of the endogenous genes, e.g. by means of overexpression. The measures for overexpression of transcription factors are known to the person skilled in the art and are also disclosed for the enzymes of Table 1 and 2 within the scope of the present invention.

Furthermore, an alteration of the activity of endogenous genes can be achieved by targeted mutagenesis of the endogenous gene copies.

An alteration of the activity of the proteins of Table 1 or 2 can also be achieved by influencing the post-translational modifications of the proteins. This can happen e.g. by regulating the activity of enzymes like kinases or phosphatases involved in the post-translational modification of the proteins by means of corresponding measures like overexpression or gene silencing.

In another embodiment, an enzyme may be improved in efficiency, or its allosteric control region destroyed such that feedback inhibition of production of the compound is prevented. Similarly, a degradative enzyme may be deleted or modified by substitution, deletion, or addition such that its degradative activity is lessened for the desired enzyme of Table 1 without impairing the viability of the cell. In each case, the overall yield or rate of production of one or more fine chemicals may be increased.

It is also possible that such alterations in the protein and nucleotide molecules of Table 1 and/or 2 may improve the production of fine chemicals other than methionine such as other sulfur containing compounds like cysteine or glutathione, other amino acids, vitamins, cofactors, nutraceuticals, nucleotides, nucleosides, and trehalose. Metabolism of any one compound is necessarily intertwined with other biosynthetic and degradative pathways within the cell, and necessary cofactors, intermediates, or substrates in one pathway are likely supplied or limited by another such pathway. Therefore, by modulating the activity of one or more of the proteins of Table 1 and/or 2, the production or efficiency of activity of another fine chemical biosynthetic or degradative pathway besides those leading to methionine synthesis may be impacted.

Enzyme expression and function may also be regulated based on the cellular level of a compound from a different metabolic process, and the cellular levels of molecules necessary for basic growth, such as amino acids and nucleotides, may critically affect the viability of the microorganism in large-scale culture. Thus, modulation of the amino acid biosynthesis enzymes of Table 1 and/or 2 such that they are no longer responsive to feedback inhibition or such that they are improved in efficiency or turnover should result in higher metabolic flux through pathways of methionine production.

These aforementioned strategies for increasing or introducing the amount and/or activity of the proteins of Table 1 and 2 are not meant to be limiting; variations on these strategies will be readily apparent to one of ordinary skill in the art.

For decreasing or suppressing or reducing the amount or content and/or activity of any one of the proteins of Table 1 and/or 2, also various strategies are available.

The expression of the endogenous enzymes of Table 1 and/or 2 can e.g. be regulated via the expression of aptamers specifically binding to the promoter sequences of the genes. Depending on the aptamers binding to stimulating or repressing promoter regions, the amount and thus, in this case, the activity of the proteins of Table 1 and/or 2 is increased or reduced.

Aptamers can also be designed in a way as to specifically bind to the enzymes themselves and to reduce the activity of the enzymes by e.g. binding to the catalytic center of the respective enzymes. The expression of aptamers is usually achieved by vector-based overexpression (see above) and is, as well as the design and the selection of aptamers, well known to the person skilled in the art (Famulok et al. (1999) Curr Top Microbiol Immunol. 243: 123-36).

Furthermore, a decrease of the amount and the activity of the endogenous enzymes of Table 1 and/or 2 can be achieved by means of various experimental measures, which are well known to the person skilled in the art. These measures are usually summarized under the terms “gene silencing”, “attenuating a gene”, “disrupting a gene” or “eliminating a gene”. For example, the expression of an endogenous gene can be silenced by transferring an above-mentioned vector, which has a DNA sequence coding for the enzyme or parts thereof in antisense order, to the organisms such as C. glutamicum and E. coli. This is based on the fact that the transcription of such a vector in the cell leads to an RNA which can hybridize with the mRNA transcribed from the endogenous gene and thereby prevents its translation.

For the expression of antisense RNA, regulatory sequences can be chosen which direct the continuous expression of the antisense RNA molecule in a variety of cell types, for instance viral promoters and/or enhancers, or regulatory sequences can be chosen which direct constitutive, tissue- or cell type-specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub H. et al. (1985) Trends in Genetics 1(1): 22-25.

In principle, the antisense strategy can be coupled with a ribozyme method. Ribozymes are catalytically active RNA sequences, which, if coupled to the antisense sequences, cleave the target sequences catalytically (Tanner et al. (1999) FEMS Microbiol Rev. 23 (3): 257-75). This can enhance the efficiency of an antisense strategy.

In plants, gene silencing may be achieved by RNA interference or a process that is known as co-suppression.

Further methods are the introduction of nonsense mutations into the endogenous gene by means of introducing RNA/DNA oligonucleotides into the organism (Zhu et al. (2000) Nat. Biotechnol. 18 (5): 555-558) or generating knockout mutants by homologous recombination (Hohn et al. (1999) Proc. Natl. Acad. Sci. USA. 96: 8321-8323.).

To create a homologous recombinant microorganism, a vector is prepared which contains at least a portion of gene coding for a protein of Table 1 or 2 into which a deletion, addition or substitution has been introduced to thereby alter, e.g., functionally disrupt, the endogenous gene.

Preferably, this endogenous gene is a C. glutamicum or E. coli gene, but it can be a homologue from a related bacterium or even from a yeast or plant source. In one embodiment, the vector is designed such that, upon homologous recombination, the endogenous gene is functionally disrupted, i.e. it no longer encodes a functional protein. Such a vector is also referred to as a “knock out” vector. Alternatively, the vector can be designed such that, upon homologous recombination, the endogenous gene is mutated or otherwise altered but still encodes a functional protein (e.g., the upstream regulatory region can be altered to thereby alter the expression of the endogenous enzyme of Table 1 or 2). In the homologous recombination vector, the altered portion of the endogenous gene is flanked at its 5′ and 3′ ends by additional nucleic acid sequences of the endogenous gene to allow for homologous recombination to occur between the exogenous gene carried by the vector and an endogenous gene in the (micro)organism. The additional flanking endogenous nucleic acid is of sufficient length for successful homologous recombination with the endogenous gene. Typically, several hundred bases to kilobases of flanking DNA (both at the 5′ and 3′ ends) are included in the vector (see e.g., Thomas, K. R., and Capecchi, M. R. (1987) Cell 51(3): 503-512 and Schafer et al. (1994) Gene 145: 69-73, for descriptions of homologous recombination vectors).

The vector is introduced into a microorganism (e.g., by electroporation) and cells in which the introduced gene has homologously recombined with the endogenous gene coding for a protein of Table 1 or 2 are selected, using art-known techniques.

In another embodiment, an endogenous gene coding for the proteins of Table 1 or 2 in a host cell is disrupted (e.g., by homologous recombination or other genetic means known in the art) such that expression of its protein product does not occur. In another embodiment, an endogenous or introduced gene coding for a protein of Table 1 or 2 in a host cell has been altered by one or more point mutations, deletions, or inversions, but still encodes a functional enzyme. In still another embodiment, one or more of the regulatory regions (e.g., a promoter, repressor, or inducer) of an endogenous gene coding for the proteins of Table 1 or 2 in a (micro)organism has been altered (e.g., by deletion, truncation, inversion, or point mutation) such that the expression of the endogenous gene is modulated. One of ordinary skill in the art will appreciate that host cells containing more than one of the genes coding for the proteins of Table 1 and 2 and protein modifications may be readily produced using the methods of the invention, and are meant to be included in the present invention.

Furthermore, gene repression (but also gene overexpression) is also possible by means of specific DNA-binding factors, e.g. factors of the zinc finger transcription factor type. Furthermore, factors inhibiting the target protein itself can be introduced into a cell. The protein-binding factors may e.g. be the above-mentioned aptamers (Famulok et al. (1999) Curr Top Microbiol Immunol. 243: 123-36).

Further protein-binding factors, whose expression in organisms causes a reduction of the amount and/or the activity of the enzymes of Table 1 or 2, may be selected from specific antibodies. The production of monoclonal, polyclonal, or recombinant specific antibodies follows standard protocols (Guide to Protein Purification, Meth. Enzymol. 182, pp. 663-679 (1990), M. P. Deutscher, ed.). The expression of antibodies is also known from the literature (Fiedler et al. (1997) Immunotechnology 3: 205-216; Maynard and Georgiou (2000) Annu. Rev. Biomed. Eng. 2: 339-76).

The mentioned techniques are well known to the person skilled in the art. Therefore, it is also well-known which sizes the nucleic acid constructs used for e.g. antisense methods must have and which complementarity, homology or identity, the respective nucleic acid sequences must have.

The term “complementarity” describes the capability of a nucleic acid molecule of hybridizing with another nucleic acid molecule due to hydrogen bonds between two complementary bases. The person skilled in the art knows that two nucleic acid molecules do not have to have a complementarity of 100% in order to be able to hybridize with each other. A nucleic acid sequence, which is to hybridize with another nucleic acid sequence, is preferably at least 40%, at least 50%, at least 60%, more preferably at least 70%, particularly preferably at least 80%, also particularly preferably at least 90%, in particular preferably at least 95% and most preferably at least 98 or 100%, respectively, complementary with said other nucleic acid sequence.

The hybridization of an antisense sequence with an endogenous mRNA sequence typically occurs in vivo under cellular conditions or in vitro. According to the present invention, hybridization is carried out in vivo or in vitro under conditions that are stringent enough to ensure a specific hybridization.

Stringent in vitro hybridization conditions are known to the person skilled in the art and can be taken from the literature (see e.g. Sambrook et al., Molecular Cloning, 3^{rd} edition 2001, Cold Spring Harbor Laboratory Press). The term “specific hybridization” refers to the case wherein a molecule preferentially binds to a certain nucleic acid sequence under stringent conditions, if this nucleic acid sequence is part of a complex mixture of e.g. DNA or RNA molecules.

The term “stringent conditions” therefore refers to conditions, under which a nucleic acid sequence preferentially binds to a complementary target sequence, but not, or at least to a significantly reduced extent, to other sequences.

Stringent conditions depend on the circumstances. Longer sequences specifically hybridize at higher temperatures. In general, stringent conditions are chosen in such a way that the hybridization temperature is about 5° C. below the melting point (Tm) of the specific sequence at a defined ionic strength and a defined pH value. Tm is the temperature (at a defined pH value, a defined ionic strength and a defined nucleic acid concentration), at which 50% of the molecules, which are complementary to a target sequence, hybridize with said target sequence. Typically, stringent conditions comprise salt concentrations between 0.01 and 1.0 M sodium ions (or ions of another salt) and a pH value between 7.0 and 8.3. The temperature is at least 30° C. for short molecules (e.g. for such molecules comprising between 10 and 50 nucleotides). In addition, stringent conditions can comprise the addition of destabilizing agents like e.g. formamide. Typical hybridization and washing buffers are of the following composition.

| | |
|---|---|
| Pre-hybridization solution: | 0.5% SDS |
| | 5x SSC |
| | 50 mM NaPO₄, pH 6.8 |
| | 0.1% Na-pyrophosphate |
| | 5x Denhardt's reagent |
| | 100 μg salmon sperm |
| Hybridization solution: | Pre-hybridization solution |
| | 1 × 10⁶ cpm/mL probe (5-10 min 95° C.) |
| 20x SSC. | 3 M NaCl |
| | 0.3 M sodium citrate |
| | ad pH 7 with HCl |
| 50x Denhardt's reagent: | 5 g Ficoll |
| | 5 g polyvinylpyrrolidone |
| | 5 g Bovine Serum Albumin |
| | ad 500 mL A. dest. |

A typical procedure for the hybridization is as follows:

| Optional: | wash Blot 30 min in 1x SSC/0.1% SDS at 65° C. | | |
|---|---|---|---|
| Pre-hybridization: | at least 2 h at 50-55° C. | | |
| Hybridization: | over night at 55-60° C. | | |
| Washing: | 5 min | 2x SSC/0.1% SDS | |
| | Hybridization temperature | | |
| | 30 min | 2x SSC/0.1% SDS | |
| | Hybridization temperature | | |
| | 30 min | 1x SSC/0.1% SDS | |
| | Hybridization temperature | | |
| | 45 min | 0.2x SSC/0.1% SDS | 65° C. |
| | 5 min | 0.1x SSC | room temperature |

The terms “sense” and “antisense” as well as “antisense orientation” are known to the person skilled in the art. Furthermore, the person skilled in the art knows how long nucleic acid molecules, which are to be used for antisense methods, must be and which degree of homology or complementarity they must have with their target sequences.

Accordingly, the person skilled in the art also knows how long nucleic acid molecules, which are used for gene silencing methods, must be. For antisense purposes complementarity over sequence lengths of 100 nucleotides, 80 nucleotides, 60 nucleotides, 40 nucleotides and 20 nucleotides may suffice. Longer nucleotide lengths will certainly also suffice. A combined application of the above-mentioned methods is also conceivable.

If, according to the present invention, DNA sequences are used, which are operatively linked in 5′-3′-orientation to a promoter active in the organism, vectors can, in general, be constructed, which, after the transfer to the organism's cells, allow the overexpression of the coding sequence or cause the suppression or competition and blockage of endogenous nucleic acid sequences and the proteins expressed therefrom, respectively.

The activity of a particular enzyme may also be reduced by over-expressing a non-functional mutant thereof in the organism. Thus, a non-functional mutant which is not able to catalyze the reaction in question, but that is able to bind e.g. the substrate or co-factor, can, by way of over-expression out-compete the endogenous enzyme and therefore inhibit the reaction. Further methods in order to reduce the amount and/or activity of an enzyme in a host cell are well known to the person skilled in the art.

Another aspect of the invention pertains to vectors, preferably expression vectors, containing a nucleic acid encoding one of the proteins of Table 1 or 2 (or portions thereof) or combinations thereof.

As used herein, the term “vector” refers to a nucleic acid molecule capable of transporting another nucleic acid to which it has been linked.

One type of vector is a “plasmid”, which refers to a circular double stranded DNA loop into which additional DNA segments can be ligated. Another type of vector is a viral vector, wherein additional DNA segments can be ligated into the viral genome.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked.

Such vectors are referred to herein as “expression vectors”.

In general, expression vectors of utility in recombinant DNA techniques are often in the form of plasmids. In the present specification, “plasmid” and “vector” can be used interchangeably as the plasmid is the most commonly used form of vector. However, the invention is intended to include also other forms of expression vectors, such as viral vectors (e.g., replication defective retroviruses, adenoviruses and adeno-associated viruses), which serve equivalent functions.

The recombinant expression vectors of the invention may comprise a nucleic acid coding for one of the proteins of Table 1 or 2 in a form suitable for expression of the respective nucleic acid in a host cell, which means that the recombinant expression vectors include one or more regulatory sequences, selected on the basis of the host cells to be used for expression, which is operatively linked to the nucleic acid sequence to be expressed.

Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell).

The term “regulatory sequence” is intended to include promoters, repressor binding sites, activator binding sites, enhancers and other expression control elements (e.g., terminators, polyadenylation signals, or other elements of mRNA secondary structure). Such regulatory sequences are described, for example, in Goeddel; Gene Expression Technology Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells. Preferred regulatory sequences are, for example, promoters such as cos-, tac-, trp-, tet-, trp-tet-, lpp-, lac-, lpp-lac-, lacIq-, T7-, T5-, T3-, gal-, trc-, ara-, SP6-, arny, SP02, e-Pp-ore PL, sod, ef-tu, groE, which are used preferably in bacteria. Additional regulatory sequences are, for example, promoters from yeasts and fungi, such as ADC1, MFa, AC, P-60, CYC1, GAPDH, TEF, rp28, ADH, promoters from plants such as CaMV/35S, SSU, OCS, lib4, usp, STLS1, B33, nos or ubiquitin- or phaseolin-promoters. It is also possible to use artificial promoters. It will be appreciated by one of ordinary skill in the art that the design of the expression vector can depend on factors such as the choice of the host cell to be transformed, the desired expression level of the protein, etc. The expression vectors of the invention can be introduced into host cells to thereby produce proteins or peptides, including fusion proteins or peptides, encoded by nucleic acids coding for the enzymes of Table 1 and/or 2.

The recombinant expression vectors of the invention can be designed for expression of the enzymes in Table 1 and/or 2 in prokaryotic or eukaryotic cells. For example, the genes for the enzymes of Table 1 and/or 2 can be expressed in bacterial cells such as C. glutamicum, B. subtilis and E. coli, insect cells (using baculovirus expression vectors), yeast and other fungal cells (see Romanos, M. A. et al. (1992) Yeast 8: 423-488; van den Hondel, C. A. M. J. J. et al. (1991) in: More Gene Manipulations in Fungi, J. W. Bennet & L. L. Lasure, eds., p. 396-428: Academic Press: San Diego; van den Hondel, C. A. M. J. J. & Punt, P. J. (1991) in: Applied Molecular Genetics of Fungi, Peberdy, J. F. et al., eds., p. 1-28, Cambridge University Press: Cambridge), algae and multicellular plant cells (see Schmidt, R. and Willmitzer, L. (1988) Plant Cell Rep. (7): 583-586). Suitable host cells are further discussed in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out with vectors containing constitutive or inducible promoters directing the expression of either fusion or non-fusion proteins.

Fusion vectors add a number of amino acids to a protein encoded by the inserted nucleic acid sequence, usually to the amino terminus of the recombinant protein but also to the C-terminus or fused within suitable regions in the proteins. Such fusion vectors typically serve three purposes: 1) to increase expression of recombinant protein; 2) to increase the solubility of the recombinant protein; and 3) to aid in the purification of the recombinant protein by providing a ligand for affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pQE (Qiagen), pGEX (Pharmacia Biotech Inc; Smith, D. B. and Johnson, K. S. (1988) Gene 67: 31-40), pMAL (New England Biolabs, Beverly, Mass.) and pRIT5 (Pharmacia, Piscataway, N.J.) which fuse glutathione S-transferase (GST), maltose E binding protein, or protein A, respectively.

Examples for C. glutamicum vectors can be found in the Handbook of Corynebacterium 2005 Eggeling, L. Bott, M., eds., CRC press USA.

Examples of suitable inducible non-fusion E. coli expression vectors include pTrc (Amann et al. (1988) Gene 69: 301-315), pLG338, pACYC184, pBR322, pUC18, pUC19, pKC30, pRep4, pHS1, pHS2, pPLc236, pMBL24, pLG200, pUR290, pIN-III113-Bl, egtll, pBdCl, and pET lld (Studier et al., Gene Expression Technology: Methods in Enzymology 185, Academic Press, San Diego, Calif. (1990) 60-89; Pouwels et al., eds. (1985) Cloning Vectors. Elsevier: New York ISBN 0 444 904018). Target gene expression from the pTrc vector relies on host RNA polymerase transcription from a hybrid trp-lac fusion promoter. Target gene expression from the pET lld vector relies on transcription from a T7 gnlO-lac fusion promoter mediated by a coexpressed viral RNA polymerase (T7gnl). This viral polymerase is supplied by host strains BL21 (DE3) or HMS174 (DE3) from a resident X prophage harboring a T7 gnl gene under the transcriptional control of the lacUV 5 promoter. For transformation of other varieties of bacteria, appropriate vectors may be selected. For example, the plasmids pIJ101, pIJ364, pIJ702 and pIJ361 are known to be useful in transforming Streptomyces, while plasmids pUB110, pC194, or pBD214 are suitable for transformation of Bacillus species. Several plasmids of use in the transfer of genetic information into Corynebacterium include pHM1519, pBL1, pSA77, or pAJ667 (Pouwels et al., eds. (1985) Cloning Vectors. Elsevier: New York IBSN 0 444 904018).

One strategy to maximize recombinant protein expression is to express the protein in host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in the bacterium chosen for expression, such as C. glutamicum (Wada et al. (1992) Nucleic Acids Res. 20: 2111-2118). Such alteration of nucleic acid sequences of the invention can be carried out using standard DNA synthesis techniques.

In another embodiment, the protein expression vector is a yeast expression vector. Examples of vectors for expression in yeast (S. cerevisiae) include pYepSec1 (Baldari, et al. (1987) Embo J. 6: 229-234), 21, pAG-1, Yep6, Yep13, pEMBLYe23, pMFa (Kurjan and Herskowitz (1982) Cell 30: 933-943), pJRY88 (Schultz et al. (1987) Gene 54: 113-123), and pYES2 (Invitrogen Corporation, San Diego, Calif.). Vectors and methods for the construction of vectors appropriate for use in other fungi, such as the filamentous fungi, include those detailed in: van den Hondel, C. A. M. J. J. & Punt, P. J. (1991) in: Applied Molecular Genetics of Fungi, J. F. Peberdy, et al., eds., p. 1-28, Cambridge University Press: Cambridge, and Pouwels et al., eds. (1985) Cloning Vectors. Elsevier: New York (ISBN 0 444 904018).

In another embodiment, the proteins of Table 1 and 2 may be expressed in unicellular plant cells (such as algae) or in plant cells from higher plants (e.g., the spermatophytes such as crop plants). Examples of plant expression vectors include those detailed in: Becker et al. (1992) Plant Mol. Biol. 20: 1195-1197; and Bevan, M. W. (1984) Nucl. Acid. Res. 12: 8711-8721, and include pLGV23, pGHlac+, pBIN19, pAK2004, and pDH51 (Pouwels et al., eds. (1985) Cloning Vectors. Elsevier: New York ISBN 0 444 904018).

For other suitable expression systems for both prokaryotic and eukaryotic cells see chapters 16 and 17 of Sambrook, J. et al. Molecular Cloning: A Laboratory Manual. 3rd ed., Cold Spring Harbor Laboratory Press, Cold Spring Harbor, N.Y., 2003.

In another embodiment, the recombinant expression vector is capable of directing expression of the nucleic acid preferentially in a particular cell type of a multicellular organism, e.g. in plant cells (e.g., tissue-specific regulatory elements are used to express the nucleic acid). Tissue-specific regulatory elements are known in the art.

Another aspect of the invention pertains to organisms or host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a protein of Table 1 and/or 2 can be expressed in bacterial cells such as C. glutamicum or E. coli, insect cells, yeast or plants. Other suitable host cells are known to those of ordinary skill in the art.

Vector DNA can be introduced into prokaryotic or eukaryotic cells via conventional transformation or transfection techniques. As used herein, the terms “transformation” and “transfection”, “conjugation” and “transduction” are intended to refer to a variety of art-recognized techniques for introducing foreign nucleic acid (e.g., linear DNA or RNA, e.g., a linearized vector or a gene construct alone without a vector) or nucleic acid in the form of a vector (e.g., a plasmid, phage, phasmid, phagemid, transposon or other DNA) into a host cell, including calcium phosphate or calcium chloride co-precipitation, DEAE-dextran-mediated transfection, lipofection, natural competence, chemical-mediated transfer, or electroporation. Suitable methods for transforming or transfecting host cells can be found in Sambrook, et al. (Molecular Cloning: A Laboratory Manual. 3rd ed., Cold Spring Harbor Laboratory Press, Cold Spring Harbor, N.Y., 2003).

“Campbell in”, as used herein, refers to a transformant of an original host cell in which an entire circular double stranded DNA molecule (for example a plasmid) is integrated into a chromosome by a single homologous recombination event (a cross-in event), and that effectively results in the insertion of a linearized version of said circular DNA molecule into a first DNA sequence of the chromosome that is homologous to a first DNA sequence of the said circular DNA molecule. The name comes from Professor Alan Campbell, who first proposed this kind of recombination. “Campbelled in” refers to the linearized DNA sequence that has been integrated into the chromosome of a “Campbell in” transformant. A “Campbell in” contains a duplication of the first homologous DNA sequence, each copy of which includes and surrounds a copy of the homologous recombination crossover point.

“Campbell out”, as used herein, refers to a cell descending from a “Campbell in” transformant, in which a second homologous recombination event (a cross-out event) has occurred between a second DNA sequence that is contained on the linearized inserted DNA of the “Campbelled in” DNA, and a second DNA sequence of chromosomal origin, which is homologous to the second DNA sequence of said linearized insert, the second recombination event resulting in the deletion (jettisoning) of a portion of the integrated DNA sequence, but, importantly, also resulting in a portion (this can be as little as a single base) of the integrated “Campbelled in” DNA remaining in the chromosome, such that compared to the original host cell, the “Campbell out” cell contains one or more intentional changes in the chromosome (for example, a single base substitution, multiple base substitutions, insertion of a heterologous gene or DNA sequence, insertion of an additional copy or copies of a homologous gene or a modified homologous gene, or insertion of a DNA sequence comprising more than one of these aforementioned examples listed above).

A “Campbell out” cell or strain is usually, but not necessarily, obtained by a counter-selection against a gene that is contained in a portion (the portion that is desired to be jettisoned) of the “Campbelled in” DNA sequence, for example the Bacillus subtilis sacB gene, which is lethal when expressed in a cell that is grown in the presence of about 5% to 10% sucrose. Either with or without a counter-selection, a desired “Campbell out” cell can be obtained or identified by screening for the desired cell, using any screenable phenotype, such as, but not limited to, colony morphology, colony color, presence or absence of antibiotic resistance, presence or absence of a given DNA sequence by polymerase chain reaction, presence or absence of an auxotrophy, presence or absence of an enzyme, colony nucleic acid hybridization, antibody screening, etc.

The term “Campbell in” and “Campbell out” can also be used as verbs in various tenses to refer to the method or process described above.

It is understood that the homologous recombination events that lead to a “Campbell in” or “Campbell out” can occur over a range of DNA bases within the homologous DNA sequence, and since the homologous sequences will be identical to each other for at least part of this range, it is not usually possible to specify exactly where the crossover event occurred. In other words, it is not possible to specify precisely which sequence was originally from the inserted DNA, and which was originally from the chromosomal DNA. Moreover, the first homologous DNA sequence and the second homologous DNA sequence are usually separated by a region of partial non-homology, and it is this region of non-homology that remains deposited in a chromosome of the “Campbell out” cell.

For practicality, in C. glutamicum, typical first and second homologous DNA sequences are usually at least about 200 base pairs in length, and can be up to several thousand base pairs in length. However, the procedure can also be adapted to work with shorter or longer sequences. For example, a length for the first and second homologous sequences can range from about 500 to 2000 bases, and obtaining a “Campbell out” from a “Campbell in” is facilitated by arranging the first and second homologous sequences to be approximately the same length, preferably with a difference of less than 200 base pairs and most preferably with the shorter of the two being at least 70% of the length of the longer in base pairs.

In order to identify and select these integrants, a gene that encodes a selectable marker (e.g., resistance to antibiotics) is generally introduced into the host cells along with the gene of interest. Preferred selectable markers include those which confer resistance to drugs, such as kanamycin, chloramphenicol, tetracyclin, G418, hygromycin and methotrexate. Nucleic acid molecules encoding a selectable marker can be introduced into a host cell on the same vector as that encoding the proteins of Table 1 and/or 2 or can be introduced on a separate vector. Cells stably transfected with the introduced nucleic acid can be identified by drug selection (e.g., cells that have incorporated the selectable marker gene will survive, while the other cells die).

In another embodiment, recombinant microorganisms can be produced which contain systems which allow for enhanced expression of the selected and/or introduced gene. Examples for altered and enhanced expression of genes in high GC organisms like C. glutamicum are described in WO 2005/059144, WO 2005/059143 and WO 2005/059093.

In another embodiment, recombinant microorganisms can be produced which contain selected systems which allow for regulated expression of the introduced gene. For example, inclusion of a gene of Table 1 or 2 on a vector placing it under control of the lac operon permits expression of the gene only in the presence of IPTG. Such regulatory systems are well known in the art.

In one embodiment, the method of the present invention further comprises isolating methionine from the medium or the host cell.

Culture media which are suitable for the method according to the present invention have been described above. If a genetically modified microorganism is used, one may use standard media which may be enriched in serine. If a wild-type microorganism is used, it has to be cultivated in a medium enriched in serine to achieve the inventive effect.

The medium is inoculated to an OD600 of 0.5-1.5 using cells grown on agar plates, such as CM plates (10 g/L glucose, 2.5 g/L NaCl, 2 g/l urea, 10 g/L polypeptone, 5 g/L yeast extract, 5 g/L meat extract, 22 g/L agar, pH 6.8 with 2M NaOH) that had been incubated at 30° C.

Inoculation of the media is accomplished by either introduction of a saline suspension of bacterial cells from CM plates or addition of a liquid preculture of the bacterium.

The incubation temperature should be in a range between 15° C. and 45° C. The temperature can be kept constant or can be altered during the experiment. The pH of the medium may be in the range of 5 to 8.5, preferably around 7.0, and can be maintained by the addition of buffers to the media. An exemplary buffer for this purpose is a potassium phosphate buffer. Synthetic buffers such as MOPS, HEPES, ACES and others can alternatively or simultaneously be used. It is also possible to maintain a constant culture pH through the addition of NaOH or NH₄OH during growth. If complex medium components such as yeast extract are utilized, the necessity for additional buffers may be reduced, due to the fact that many complex compounds have high buffer capacities. If a fermentor is utilized for culturing the micro-organisms, the pH can also be controlled using gaseous ammonia.

The incubation time is usually in a range from several hours to several days. This time is selected in order to permit the maximal amount of product to accumulate in the broth without letting the microorganisms accumulate to such densities that cell death is induced.

The disclosed growth experiments can be carried out in a variety of vessels, such as microtiter plates, glass tubes, glass flasks or glass or metal fermentors of different sizes. For screening a large number of clones, the microorganisms should be cultured in microtiter plates, glass tubes or shake flasks, either with or without baffles. Preferably 100 mL shake flasks are used, filled with 10% (by volume) of the required growth medium. The flasks should be shaken on a rotary shaker (amplitude 25 mm) using a speed-range of 100-300 rpm. Evaporation losses can be diminished by the maintenance of a humid atmosphere; alternatively, a mathematical correction for evaporation losses should be performed.

If genetically modified clones are tested, an unmodified control clone or a control clone containing the basic plasmid without any insert should also be tested.

After the cultivation, the bacteria are harvested by centrifugation under conditions which leave the bacterial cells intact.

The broth after fermentation can be treated by adding acids or bases to obtain a suitable pH-value that allows binding of the methionine to a matrix which can consist of an anion- or a cation-exchange matrix. In addition or alternatively to the aforementioned measures the broth either with or without biomass can be concentrated by evaporation and/or cooled to temperatures between 20-0° C. Under these conditions the methionine in the fermentation broth can be crystallized since the solubility of methionine at pH values between 3 and 9 in water is low and depends on the temperature of the solvent. In addition or alternatively to all mentioned measures methionine accumulated in the broth can be dried by methods such as spray drying or other drying methods either with or without biomass. In all these cases a material is being produced that can contain 5-99% methionine by weight, preferably 15-99% methionine by weight, more preferably 30-99% methionine by weight, even more preferably 50-99% methionine by weight and most preferably 70-99% methionine by weight.

Although the present invention has been described with reference to Corynebacterium glutamicum and the production of L-methionine, it should be pointed out that the present invention can also be applied to other microorganisms and to the production of other amino acids.

In addition, it should be pointed out that “comprising” does not exclude any other elements or steps and that “one” does not exclude a plural number. Furthermore, it should be pointed out that the characteristics or steps which have been described with reference to one of the above embodiments can also be used in combination with other characteristics or steps of other embodiments described above.

The invention is further illustrated by the following examples, which should not be construed as limiting. The contents of all references, patent applications, patents, published patent applications, tables, appendices and the sequences cited throughout this application are hereby incorporated by reference.

### EXAMPLES

Bacterial strain. Corynebacterium glutamicum ATCC 13032 (wild-type) was obtained from the American Type Culture Collection (Manassas, Va., USA). The knockout mutant for MbcR was constructed as follows:

C. glutamicum M1840 was a ΔMcbR strain derived from the wild type ATCC13032 (Rey et al., 2003, vide supra). ATCC 13032 was transformed with the plasmid pH430 (SEQ ID No. 1) and “Campbelled in” in to yield “Campbell in” strains. “Campbell in” strains were then “Campbelled out” to yield “Campbell out” strain M1840, which contains a deletion of the McbR gene.

Medium. M1840 was grown in CG12½ minimal medium. This medium was prepared by mixing different stock solutions (solutions 1-8).

Solution 1: 25.0 g glucose
ad 100 ml H₂O, autoclave

### Solution 2: 4.0 g KH₂PO₄

16.0 g K₂HPO₄
adjust the pH to 7.0 with NaOH, ad 695 ml H₂O, autoclave

### Solution 3: 10.0 g (NH₄)₂SO₄

adjust the pH to 7.0 with NaOH, ad 200 ml H₂O, autoclave

### Solution 4: 2.5 g MgSO₄×7H₂O

ad 10 ml H₂O, filtrate

### Solution 5: 0.1 g CaCl₂

ad 10 ml H₂O, filtrate
   Solution 6: 0.3 g 3,4-dihydroxy benzoic acid
adjust the pH to 12.0 with NaOH, ad 10 ml H₂O, filtrate
   Solution 7: 50 μl vitamin B12 (stock solution: 100 μg/ml)
0.015 g thiamine
10 μl pyridoxal phosphate (stock solution: 0.1 mg/ml)
5 ml biotin (stock solution: 1 mg/ml)
ad 50 ml H₂O, filtrate

### Solution 8: 0.5 g FeSO₄×7H₂O

0.5 g MnSO₄×H₂O
0.1 g ZnSO₄×7H₂O
500 μl CuSO₄×5H₂O (stock solution: 0.02 g/ml)
50 μl NiCl₂×6H₂O (stock solution: 0.02 g/ml)
50 μl Na₆Mo₇O₂₄×2H₂O (stock solution: 0.02 g/ml)
adjust the pH to 1 with HCl, ad 50 ml H₂O, filtrate

1 l of CG12½ minimal medium was prepared by mixing 80 ml of solution 1, 695 ml of solution 2, 200 ml of solution 3 and 1 ml of each of solutions 4 to 8. Furthermore, 20 ml sterile water were added.

For the medium enriched in serine 10 mM serine were added to the CG12½ minimal medium.

Growth conditions. The cells were maintained on plates at 30° C. Precultures were grown over night in 250 mL baffled shake flasks with 25 mL rich liquid medium. The cells were harvested by centrifugation (2 min, 10000 g, 4° C.), washed twice with 0.9% NaCl and used for inoculation in the second preculture on CG12½ minimal medium. The second preculture was harvested as described above and used as starter of the main cultivations, carried out on CG12½ minimal medium. The cells were harvested at late exponential phase. Other experiments were carried out in 500 mL baffled shake flasks in 50 mL medium on a rotary shaker (250 rpm, 30° C., shaking radius 2.5 cm).

Cell extraction and quantification of amino acids. The cells were extracted as described previously (Wittmann et al. (2004) Anal. Biochem. 327: 135-139). The quantification of methionine was performed by HPLC (Agilent 1100, Waldbronn, Germany). Before analysis, all samples were diluted 1:10 with a 225 μM aqueous solution of α-amino butyric acid using an analytical balance. α-amino butyric acid served as an internal standard in the quantification. The amino acids were detected using a fluorescence detector (340 nm excitation, 450 nm emission; Agilent, Waldbronn, Germany). For this purpose, a precolumn derivatization with o-phthaldialdehyde was performed (Roth (1971) Anal. Chem. 43: 880-882).

### Results:

Upon addition of serine to the minimal medium the intracellular methionine concentration increased from 0.42_{±0.06} g/dry mass for cells grown in non-enriched medium to 0.71_{±0.12} g/dry mass for cells grown in medium enriched with serine.

SeqID No. 1:
>pH430
tcgagctctccaatctccactgaggtacttaatccttccggggaattcgg
gcgcttaaatcgagaaattaggccatcaccttttaataacaatacaatga
ataattggaataggtcgacacctttggagcggagccggttaaaattggca
gcattcaccgaaagaaaaggagaaccacatgcttgccctaggttggatta
catggatcattattggtggtctagctggttggattgcctccaagattaaa
ggcactgatgctcagcaaggaattttgctgaacatagtcgtcggtattat
cggtggtttgttaggcggctggctgcttggaatcttcggagtggatgttg
ccggtggcggcttgatcttcagcttcatcacatgtctgattggtgctgtc
attttgctgacgatcgtgcagttcttcactcggaagaagtaatctgcttt
aaatccgtagggcctgttgatatttcgatatcaacaggccttttggtcat
tttggggtggaaaaagcgctagacttgcctgtggattaaaactatacgaa
ccggtttgtctatattggtgttagacagttcgtcgtatcttgaaacagac
caacccgaaaggacgtggccgaacgtggctgctagctaatccttgatggt
ggacttgctggatctcgattggtccacaacatcagtcctcttgagacggc
tcgcgatttggctcggcagttgttgtcggctccacctgcggactactcaa
tttagtttcttcattttccgaaggggtatcttcgttgggggaggcgtcga
taagccccttctttttagctttaacctcagcgcgacgctgctttaagcgc
tgcatggcggcgcggttcatttcacgttgcgtttcgcgcctcttgttcgc
gatttctttgcgggcctgttttgcttcgttgatttcggcagtacgggttt
tggtgagttccacgtttgttgcgtgaagcgttgaggcgttccatggggtg
agaatcatcagggcgcggtttttgcgtcgtgtccacaggaagatgcgctt
ttctttttgttttgcgcggtagatgtcgcgctgctctaggtggtgcactt
tgaaatcgtcggtaagtgggtatttgcgttccaaaatgaccatcatgatg
attgtttggaggagcgtccacaggttgttgctgacgcgtcatatgactag
ttcggacctagggatatcgtcgacatcgatgctcttctgcgttaattaac
aattgggatcctctagacccgggatttaaatcgctagcgggctgctaaag
gaagcggaacacgtagaaagccagtccgcagaaacggtgctgaccccgga
tgaatgtcagctactgggctatctggacaagggaaaacgcaagcgcaaag
agaaagcaggtagcttgcagtgggcttacatggcgatagctagactgggc
ggttttatggacagcaagcgaaccggaattgccagctggggcgccctctg
gtaaggttgggaagccctgcaaagtaaactggatggctttcttgccgcca
aggatctgatggcgcaggggatcaagatctgatcaagagacaggatgagg
atcgtttcgcatgattgaacaagatggattgcacgcaggttctccggccg
cttgggtggagaggctattcggctatgactgggcacaacagacaatcggc
tgctctgatgccgccgtgttccggctgtcagcgcaggggcgcccggttct
ttttgtcaagaccgacctgtccggtgccctgaatgaactgcaggacgagg
cagcgcggctatcgtggctggccacgacgggcgttccttgcgcagctgtg
ctcgacgttgtcactgaagcgggaagggactggctgctattgggcgaagt
gccggggcaggatctcctgtcatctcaccttgctcctgccgagaaagtat
ccatcatggctgatgcaatgcggcggctgcatacgcttgatccggctacc
tgcccattcgaccaccaagcgaaacatcgcatcgagcgagcacgtactcg
gatggaagccggtcttgtcgatcaggatgatctggacgaagagcatcagg
ggctcgcgccagccgaactgttcgccaggctcaaggcgcgcatgcccgac
ggcgaggatctcgtcgtgacccatggcgatgcctgcttgccgaatatcat
ggtggaaaatggccgcttttctggattcatcgactgtggccggctgggtg
tggcggaccgctatcaggacatagcgttggctacccgtgatattgctgaa
gagcttggcggcgaatgggctgaccgcttcctcgtgctttacggtatcgc
cgctcccgattcgcagcgcatcgccttctatcgccttcttgacgagttct
tctgagcgggactctggggttcgaaatgaccgaccaagcgacgcccaacc
tgccatcacgagatttcgattccaccgccgccttctatgaaaggttgggc
ttcggaatcgttttccgggacgccggctggatgatcctccagcgcgggga
tctcatgctggagttcttcgcccacgctagcggcgcgccggccggcccgg
tgtgaaataccgcacagatgcgtaaggagaaaataccgcatcaggcgctc
ttccgcttcctcgctcactgactcgctgcgctcggtcgttcggctgcggc
gagcggtatcagctcactcaaaggcggtaatacggttatccacagaatca
ggggataacgcaggaaagaacatgtgagcaaaaggccagcaaaaggccag
gaaccgtaaaaaggccgcgttgctggcgtttttccataggctccgccccc
ctgacgagcatcacaaaaatcgacgctcaagtcagaggtggcgaaacccg
acaggactataaagataccaggcgtttccccctggaagctccctcgtgcg
ctctcctgttccgaccctgccgcttaccggatacctgtccgcctttctcc
cttcgggaagcgtggcgctttctcatagctcacgctgtaggtatctcagt
tcggtgtaggtcgttcgctccaagctgggctgtgtgcacgaaccccccgt
tcagcccgaccgctgcgccttatccggtaactatcgtcttgagtccaacc
cggtaagacacgacttatcgccactggcagcagccactggtaacaggatt
agcagagcgaggtatgtaggcggtgctacagagttcttgaagtggtggcc
taactacggctacactagaaggacagtatttggtatctgcgctctgctga
agccagttaccttcggaaaaagagttggtagctcttgatccggcaaacaa
accaccgctggtagcggtggtttttttgtttgcaagcagcagattacgcg
cagaaaaaaaggatctcaagaagatcctttgatcttttctacggggtctg
acgctcagtggaacgaaaactcacgttaagggattttggtcatgagatta
tcaaaaaggatcttcacctagatccttttaaaggccggccgcggccgcca
tcggcattttcttttgcgtttttatttgttaactgttaattgtccttgtt
caaggatgctgtctttgacaacagatgttttcttgcctttgatgttcagc
aggaagctcggcgcaaacgttgattgtttgtctgcgtagaatcctctgtt
tgtcatatagcttgtaatcacgacattgtttcctttcgcttgaggtacag
cgaagtgtgagtaagtaaaggttacatcgttaggatcaagatccattttt
aacacaaggccagttttgttcagcggcttgtatgggccagttaaagaatt
agaaacataaccaagcatgtaaatatcgttagacgtaatgccgtcaatcg
tcatttttgatccgcgggagtcagtgaacaggtaccatttgccgttcatt
ttaaagacgttcgcgcgttcaatttcatctgttactgtgttagatgcaat
cagcggtttcatcacttttttcagtgtgtaatcatcgtttagctcaatca
taccgagagcgccgtttgctaactcagccgtgcgttttttatcgctttgc
agaagtttttgactttcttgacggaagaatgatgtgcttttgccatagta
tgctttgttaaataaagattcttcgccttggtagccatcttcagttccag
tgtttgcttcaaatactaagtatttgtggcctttatcttctacgtagtga
ggatctctcagcgtatggttgtcgcctgagctgtagttgccttcatcgat
gaactgctgtacattttgatacgtttttccgtcaccgtcaaagattgatt
tataatcctctacaccgttgatgttcaaagagctgtctgatgctgatacg
ttaacttgtgcagttgtcagtgtttgtttgccgtaatgtttaccggagaa
atcagtgtagaataaacggatttttccgtcagatgtaaatgtggctgaac
ctgaccattcttgtgtttggtcttttaggatagaatcatttgcatcgaat
ttgtcgctgtctttaaagacgcggccagcgtttttccagctgtcaataga
agtttcgccgactttttgatagaacatgtaaatcgatgtgtcatccgcat
ttttaggatctccggctaatgcaaagacgatgtggtagccgtgatagttt
gcgacagtgccgtcagcgttttgtaatggccagctgtcccaaacgtccag
gccttttgcagaagagatatttttaattgtggacgaatcaaattcagaaa
cttgatatttttcatttttttgctgttcagggatttgcagcatatcatgg
cgtgtaatatgggaaatgccgtatgtttccttatatggcttttggttcgt
ttctttcgcaaacgcttgagttgcgcctcctgccagcagtgcggtagtaa
aggttaatactgttgcttgttttgcaaactttttgatgttcatcgttcat
gtctccttttttatgtactgtgttagcggtctgcttcttccagccctcct
gtttgaagatggcaagttagttacgcacaataaaaaaagacctaaaatat
gtaaggggtgacgccaaagtatacactttgccctttacacattttaggtc
ttgcctgctttatcagtaacaaacccgcgcgatttacttttcgacctcat
tctattagactctcgtttggattgcaactggtctattttcctcttttgtt
tgatagaaaatcataaaaggatttgcagactacgggcctaaagaactaaa
aaatctatctgtttcttttcattctctgtattttttatagtttctgttgc
atgggcataaagttgcctttttaatcacaattcagaaaatatcataatat
ctcatttcactaaataatagtgaacggcaggtatatgtgatgggttaaaa
aggatcggcggccgctcgatttaaatc

1. A process for the preparation of L-methionine in a microorganism comprising the following steps:
cultivating the microorganism wherein the amount of serine available for the metabolism of the microorganism is increased; and
isolating L-methionine,
wherein the microorganism is cultivated in a medium enriched in serine.

2. The process according to claim 1, wherein the concentration of serine added to the medium is from 0.1 mM to 100 mM.

3. The process according to claim 1, wherein the content and/or the biological activity of one or more enzymes involved in serine synthesis and/or the content and/or the biological activity of one or more enzymes involved in methionine synthesis is increased compared to the wild-type microorganism.

4. (canceled)

5. The process according to claim 3, wherein the enzyme involved in serine synthesis is selected from the group consisting of D-3-phosphoglycerate dehydrogenase (SerA), phosphoserine phosphatase (SerB) and phosphoserine aminotransferase (SerC).

6. The process according to any of claims 3, wherein the enzyme involved in serine synthesis is modified to reduce or prevent the feedback-inhibition by L-serine.

7. The process according to claim 6, wherein the enzyme being feedback inhibited is D-3-phosphoglycerate dehydrogenase (SerA).

8. The process according to claim 1, wherein the content and/or the biological activity of one or more enzymes involved in serine degradation to pyruvate is reduced compared to the wild-type microorganism.

9. The process according to claim 8, wherein the gene which codes for the enzyme involved in serine degradation to pyruvate is disrupted and preferably eliminated.

10. The process according to claim 8, wherein the enzyme is serine dehydratase (sdaA).

11. The process according to claim 1, wherein the content and/or the biological activity of one or more proteins involved in serine export is reduced compared to the wild-type microorganism.

12. The process according to claim 11, wherein the gene which codes for the protein involved in serine export is disrupted and preferably eliminated.

13. The process according to claim 11, wherein the protein is ThrE.

14. The process according to claim 1, wherein the content and/or the biological activity of one or more enzymes involved in the conversion of serine to methyl tetrahydrofolate is increased compared to the wild-type microorganism.

15. The process according to claim 14, wherein the enzyme involved in the conversion of serine to methyl tetrahydrofolate is selected from the group consisting of serine hydroxymethyltransferase (SHMT) and methylene tetrahydrolate reductase (MetF).

16. The process according to claim 3, wherein the enzyme involved in methionine synthesis is selected from the group consisting of aspartokinase (lysC), homoserine dehydrogenase (hom), homoserine-O-acetyltransferase (MetA), O-acetylhomoserine sulfhydrolase (MetZ), cob(I)alamin dependent methionine synthase I (MetH) and cob(I)alamin independent methionine synthase II (MetE).

17. The process according to claim 1, wherein the content and/or the biological activity of one or more transcriptional regulator proteins is reduced compared to the wild-type microorganism.

18. The process according to claim 17, wherein the transcriptional regulator protein is McbR.

19. The process according to claim 1, wherein the microorganism is selected from the group consisting of coryneform bacteria, mycobacteria, streptomycetaceae, salmonella, Escherichia coli, Shigella, Bacillus, Serratia and Pseudomonas.

20. The process according to claim 19, wherein the microorganism is Corynebacterium glutamicum, Escherichia coli, or Bacillus subtilis.

21. The process according to claim 1, wherein L-methionine is concentrated in the medium or in the cells of the microorganism.

22. The process for the preparation of L-methionine containing feedstuffs additive from fermentation broths, comprising the following steps:
cultivating the microorganism wherein the amount of serine available for the metabolism of the microorganism is increased;
removing water from the L-methionine containing fermentation broth;
removing an amount of 0 to 100 wt. % of the biomass formed during fermentation; and
drying the fermentation broth to obtain the animal feedstuffs additive in powder or granule form,
wherein the microorganism is cultivated in a medium enriched in serine.

23. The process according to claim 22, wherein the concentration of serine added to the medium is from 0.1 mM to 100 mM.

24. The process according to claim 22, wherein the content and/or the biological activity of one or more enzymes involved in serine synthesis and/or the content and/or the biological activity of one or more enzymes involved in methionine synthesis is increased compared to the wild-type microorganism.

25. (canceled)

26. The process according to claim 24, wherein the enzyme involved in serine synthesis is selected from the group consisting of D-3-phosphoglycerate dehydrogenase (SerA), phosphoserine phosphatase (SerB) and phosphoserine aminotransferase (SerC),

27. The process according to any of claims 24, wherein the enzyme involved in serine synthesis is modified to reduce or prevent the feedback-inhibition by L-serine.

28. The process according to claim 27, wherein the enzyme being feedback inhibited is D-3-phosphoglycerate dehydrogenase (SerA).

29. The process according to any of claims 22, wherein the content and/or the biological activity of one or more enzymes involved in serine degradation to pyruvate is reduced compared to the wild-type microorganism.

30. The process according to claim 29, wherein the gene which codes for the enzyme involved in serine degradation to pyruvate is disrupted and preferably eliminated.

31. The process according to claim 29, wherein the enzyme is serine dehydratase (sdaA).

32. The process according to claim 22, wherein the content and/or the biological activity of one or more proteins involved in serine export is reduced compared to the wild-type microorganism.

33. The process according to claim 32, wherein the gene which codes for the protein involved in serine export is disrupted and preferably eliminated.

34. The process according to claim 32, wherein the protein is ThrE.

35. The process according to claim 22, wherein the content and/or the biological activity of one or more enzymes involved in the conversion of serine to methyl tetrahydrofolate is increased compared to the wild-type microorganism.

36. The process according to claim 35, wherein the enzyme involved in the conversion of serine to methyl tetrahydrofolate is selected from the group consisting of serine hydroxymethyltransferase (SHMT) and methylene tetrahydrolate reductase (MetF).

37. The process according to claim 24, wherein the enzyme involved in methionine synthesis is selected from the group consisting of aspartokinase (lysC), homoserine dehydrogenase (hom), homoserine-O-acetyltransferase (MetA), O-acetylhomoserine sulfhydrolase (MetZ), cob(I)alamin dependent methionine synthase I (MetH) and cob(I)alamin independent methionine synthase II (MetE).

38. The process according to claim 22, wherein the content and/or the biological activity of one or more transcriptional regulator proteins is reduced compared to the wild-type microorganism.

39. The process according to claim 38, wherein the transcriptional regulator protein is McbR.

40. The process according to claim 22, wherein the microorganism is selected from the group consisting of coryneform bacteria, mycobacteria, streptomycetaceae, salmonella, Escherichia coli, Shigella, Bacillus, Serratia and Pseudomonas.

41. The process according to claim 40, wherein the microorganism is Corynebacterium glutamicum, Escherichia coli, or Bacillus subtilis.

42. A L-methionine overproducing microorganism,
wherein the content and/or the biological activity of one or more enzymes involved in serine synthesis is increased compared to the wild-type microorganism; and
optionally the content and/or the biological activity of one or more enzymes involved in serine degradation to pyruvate is reduced compared to the wild-type microorganism; and
optionally the content and/or the biological activity of one or more proteins involved in serine export is reduced compared to the wild-type microorganism; and
optionally the content and/or the biological activity of one or more enzymes involved in the conversion of serine to methyl tetrahydrofolate is increased compared to the wild-type microorganism;
and wherein the content and/or the biological activity of one or more enzymes involved in methionine synthesis is increased compared to the wild-type microorganism; and
optionally the content and/or the biological activity of one or more transcriptional regulator proteins is reduced compared to the wild-type microorganism.

43. The microorganism according to claim 42, wherein the enzyme involved in serine synthesis is selected from the group consisting of D-3-phosphoglycerate dehydrogenase (SerA), phosphoserine phosphatase (SerB) and phosphoserine aminotransferase (SerC).

44. The microorganism according to claim 42, wherein the enzyme involved in serine synthesis is modified to reduce or prevent the feedback-inhibition by L-serine.

45. The microorganism according to claim 42, wherein the enzyme involved in serine degradation to pyruvate is sdaA.

46. The microorganism according to claim 42, wherein the protein involved in serine export is ThrE.

47. The microorganism according to claim 42, wherein the enzyme involved in the conversion of serine to methyl tetrahydrofolate is selected from the group consisting of serine hydroxymethyltransferase (SHMT) and methylene tetrahydrolate reductase (MetF).

48. The microorganism according to claim 42, wherein the enzyme involved in methionine synthesis is selected from the group consisting of aspartokinase (lysC), homoserine dehydrogenase (hom), homoserine-O-acetyltransferase (MetA), O-acetylhomoserine sulfhydrolase (MetZ), cob(I)alamin dependent methionine synthase I (MetH) and cob(I)alamin independent methionine synthase II (MetE).

49. The microorganism according to claim 42, wherein the transcriptional regulator protein is McbR.

50. The microorganism according to claim 42, wherein the microorganism is selected from the group consisting of coryneform bacteria, mycobacteria, streptomycetaceae, salmonella, Escherichia coli, Shigella, Bacillus, Serratia and Pseudomonas.

51. The microorganism according to claim 50, wherein the microorganism is Corynebacterium glutamicum, Escherichia coli, or Bacillus subtilis.

52. A method of making L-methionine which comprises culturing the microorganism according to claim 42.
